(19) United States
(12) Patent Application Publication
(10) Pub. No.: US 2026/0201052 A1
(43) Pub. Date: Jul. 16, 2026

(54) CD47 BLOCKING AGENT AND ANTI-CD20 / ANTI-CD3 BISPECIFIC ANTIBODY COMBINATION THERAPY

(71) Applicants: Pfizer Inc., New York, NY (US); Hoffmann-La Roche Inc., Little Falls, NJ (US)

(72) Inventors: Ingmar Bruns, Cambridge, MA (US); Victor Ruberio Lincha, San Diego, CA (US); Linda Maria Lundberg, Binningen (CH); James Christopher Relf, Barnet (GB); Anita Scheuber, Boston, MA (US); Diane Dan Wang, San Diego, CA (US); Yibo Wang, Ellicott City, MD (US)

(73) Assignees: Pfizer Inc., New York, NY (US); Hoffmann-La Roche Inc., Little Falls, NJ (US)

(21) Appl. No.: 19/136,536

(22) PCT Filed: Dec. 8, 2023
(86) PCT No.: PCT/US2023/083080

§ 371 Date: Jun. 6, 2025

Related U.S. Application Data
(60) Provisional application No. 63/386,767, filed on Dec. 9, 2022.
(60) Provisional application No. 63/501,775, filed on May 12, 2023.

(51) Int. Cl. C07K 16/28; A61K 38/00; A61K 39/00; C07K 14/705

(52) U.S. Cl. CPC C07K 16/2887; C07K 14/70596; C07K 16/2809; A61K 38/00; A61K 2039/545; C07K 2317/31; C07K 2317/565; C07K 2319/30

(57) ABSTRACT

Dosing regimens and methods for administering combination therapies combining a CD47 blocking agent and an anti-CD20/anti-CD3 bispecific antibody are provided. The dosing regimens and methods may further include additional therapeutic agents.

### REFERENCE TO AN ELECTRONIC SEQUENCE LISTING

The contents of the electronic sequence listing (P113970014WO00-SEQ-LJG.xml; Size: 29,669 bytes; and Date of Creation: Dec. 5, 2023) is herein incorporated by reference in its entirety.

### BACKGROUND

Cancer cells are targeted for destruction by antibodies that bind to cancer cell antigens, and through recruitment and activation of macrophages by way of Fc receptor binding to the Fc portion of that antibody. Binding between CD47 on cancer cells and SIRPalpha (SIRPa) on macrophages transmits a “don't eat me” signal that enables many tumour cells to escape destruction by macrophages. It has been shown that inhibition of the CD47/SIRPa interaction (CD47 blockade) will allow macrophages to “see” and destroy the target CD47+ cancer cell. The use of SIRPa to treat cancer by CD47 blockade is described in WO 2010/130053, incorporated herein by reference. International Patent Application Publication No. WO 2014/094122, incorporated by reference in its entirety, describes a protein drug that inhibits the interaction between CD47 and SIRPa. This CD47 blockade drug is a form of human SIRPa that incorporates a particular region of its extracellular domain linked with a particularly useful form of an IgG-based Fc region. In this form, the SIRPaFc drug shows dramatic effects on the viability of cancer cells that present with a CD47+ phenotype.

Another therapeutic approach for targeting cancer cells for destruction are bispecific antibodies that are directed to both a T cell antigen and a tumor cell antigen. Bispecific antibodies that simultaneously bind to T cells and tumor cell antigens can lead to T-cell activation, proliferation, and tumor cell death. Simultaneous binding of bispecific antibodies to CD3 on T cells and target antigens on tumor cells brings the T cells into close proximity with target tumor cells, mimicking an immunological synapse and leading to T-cell-mediated killing of the tumor cells.

Bispecific antibodies which target the antigen CD20 are under development for the treatment of B-cell lymphomas. CD20 is expressed on the surface of the majority of B cells. Exemplary anti-CD20/anti-CD3 bispecific antibodies include mosunetuzumab (Roche), glofitamab (Roche), odronextamab (Regeneron), epcoritamab (Genmab/Abbvie), plamotamab (Xencor), and IGM-2323 (IGM Biosciences).

Diffuse large B cell lymphoma (DLBCL) is the most common type of lymphoma encompassing around 30% to 40% of the newly diagnosed cases of non-Hodgkin Lymphoma (NHL). R-CHOP (rituximab, cyclophosphamide, doxorubicin, vincristine, prednisolone) has been the standard treatment for newly diagnosed patients for over 2 decades curing approximately 60% of the patients. In frail and older patients mini R-CHOP (a reduced intensity regimen for patients over 80 years old) may be used as first line treatment. Patients refractory to or relapsing after first line treatment may proceed to induction chemotherapy treatment (e.g., R-ICE or R-DHAP) followed by autologous stem cell transplant (ASCT). Patients not eligible to transplant due to age or co-morbidities may proceed to less invasive treatments like R-GemOx (rituximab, gemcitabine and oxaliplatin). Tafasitamab, a CD19 monoclonal antibody with a modified fragment crystallizable (Fc)-domain, in combination with lenalidomide recently received accelerated/conditional approval for the treatment of patients with RR DLBCL not eligible for ASCT.

Polatuzumab vedotin (pola), an antibody drug conjugate targeting CD79b on the B cells surface, in combination with bendamustine and rituximab (BR) received accelerated/conditional approval in R/R DLBCL after at least 2 prior lines of therapies. Approval of this combination was based on a randomized trial of 80 patients (1:1 randomization to pola-BR vs BR). Reported response rate at end of treatment in the pola-BR arm was significantly higher than the control arm (40.0% v 17.5%; P=0.026); PFS and OS were also significantly higher in the experimental arm (HR=0.36, 95% CI, 0.21-0.63; P<0.001 and HR=0.42; 95% CI, 0.24 to 0.75; P=0.002, respectively) (Sehn L H et al, J Clin Oncol. 2020; 38 (2): 155-165). A confirmatory phase 3 trial run in untreated DLBCL patients with intermediated or high-risk disease compared the progression free survival of R-CHOP versus a modified R-CHOP regimen where polatuzumab vedotin was administered instead of vincristine (pola-R-CHP). In this study, after a median follow up of 28.2 months, the percentage of patients surviving without progression was significantly higher in the pola-R-CHP group than in the R-CHOP group (76.7% [95% confidence interval (CI), 72.7 to 80.8] vs. 70.2% [95% CI, 65.8 to 74.6] at 2 years) (Tilly H et al, N Engl J Med. 2022; 386 (4): 351-363). This treatment was recently approved in the EU for frontline DLBCL making it the first approved combination in this setting in 20 years.

Chimeric antigen receptor (CAR)-T cell therapies (like axicabtagene ciloleucel, tisagenlecleucel and lisocabtagene maraleuce) are also active new treatment options for patients with R/R DLBCL. Between them, for example, activity of axicabtagene ciloleucel, an autologous anti-CD19 CAR-T cell therapy, was explored in the ZUMA-1 trial. In this study 82% of the patients had an objective response, 58% achieved a complete response and the median PFS was 5.9 months. This led to the approval in the US as a new treatment option for patients with R/R DLBCL that received at least 2 prior lines of therapy. In addition, results from the ZUMA-7 trial (NCT03391466) led to the recent approval in the US of axicabtagene ciloleucel in patients with DLBCL refractory to first line chemoimmunotherapy or with relapse within 12 months of first line chemoimmunotherapy.

Unfortunately CAR-T cell therapy still presents challenges for patients' access, including being available only at major academic institutions, turn-around time required for leukapheresis, and presenting with significant toxicities (48% of the patients reported Grade 3 or higher adverse events, including 11% cytokine release syndrome and 32% neurological events) which are currently limiting their use (Goldfinger M & Cooper D L, Lymphoma Myeloma Leuk. 2022; 22 (3): 140-148). Therefore, new treatments are urgently needed as this constitutes an area of unmet medical need with currently limited available treatment options.

Bispecific antibodies are emerging as possible alternative therapy to CAR-T.

Glofitamab is a human IgG1-bispecific antibody targeting CD20 expressed on the surface of B cells and CD3e chain expressed on the surface of T cells (Bacac et al, Clin Cancer Res. 2018; 24 (19): 4785-4797; Mihalyova J et al, Int J Mol Sci. 2021; 22 (21)). By means of binding to CD20 on tumor cells, glofitamab induces tumor cell lysis while achieving T-cell activation, proliferation, and cytokine release by simultaneously binding to CD3 on the T cell-receptor complex. Unique to its structure, glofitamab contains two CD20 and a single CD3 binding domain (2:1 design), binding to human CD20 on B cells through two fragment antigen-binding (Fab) domains, and to the human CD3e of the TCR complex on T cells through a single Fab domain. Preliminary monotherapy safety and efficacy was evaluated in a phase 1/2 study (Study NP30179 (NCT03075696)). In part 1 of this study 171 patients with R/R B-NHL received a single dose of obinutuzumab 7 days prior to treatment with glofitamab to reduce the risk of serious cytokine-release syndrome (CRS) by depleting peripheral and tissue-based B cells (Hutchings M et al, J Clin Oncol (2021) 39 (18): 1959-1970). In addition, the study investigated a step-up dosing regimen with the intent to reduce the risk of CRS, a primarily first dose effect with frequency and severity associated with higher initial glofitamab doses. The recommended phase 2 dose (RP2D) from the part 1 of the study was 2.5 mg on C1D1, 10 mg on C1D8 and 30 mg on Day 1 of Cycle 2-Cycle 12. Additional 155 patients with R/R DLBCL who had received ≥2 prior regimens including ≥1 anti-(a) CD20 Ab and ≥1 anthracycline were treated in the expansion part of the study (Dickinson et al., European Hematology Association (EHA) Annual Meeting, June 2022, Vienna, Austria). In this part of the study reported ORR was 51.6% [95% CI: 43.5%, 59.7%], CR rate was 39.4% [95% CI: 31.6%, 47.5%] and median PFS was 4.9 months [95% CI: 3.4, 8.1] with a median duration of FU of 12.6 months (range: 0-22). The responses were highly durable with median DoR and DoCR of 18.4 months (95% CI: 13.7, NE) and NE months (95% CI: 16.8, NE), respectively. CRS occurred in 63% of the patents (with Grade 3 and 4 reported in 3.9% of the patients). Other Grade≥3 TEAEs of interest included neutropenia (26.6%), infections (14.9%), neurological AEs (3.2%), and febrile neutropenia/tumor flare/ICANS (2.6% each).

Increased anti-tumor activity in R/R DLBCL has been seen when glofitamab was combined with polatuzumab vedotin in a phase1b/2 study (Hutchings M et al, Blood. 2021; 138:626). The ORR was 73% and CR was 51.5%. For this combination the most frequent AE reported was CRS in 55% of the patients, however no Grade 3 was observed. Although no ICANS were reported, Grade 1 peripheral neuropathy was reported in 11% of the patients due to polatuzumab vedotin.

Preclinical studies demonstrate that the CD47 blockade agent TTI-622 (CD47-binding domain of human SIRPa linked to the Fc region of human IgG4) induces macrophage-mediated phagocytosis of different malignant cell lines, including DLBCL cells, decreases tumor growth and improves survival in a DLBCL xenograft tumor model. An additional preclinical study reported that anti-CD47 mediated phagocytosis of tumor cells is followed by activation of CD8+ T cells which results in antitumor activity (Tseng D, et al. Proc Natl Acad Sci USA, 2013; 110 (27): 11103-8).

The CD47 blockade and anti-CD20/anti-CD3 bispecific antibody approaches in anti-cancer drug development and treatment shows great promise for various types of cancer, including B cell lymphomas such as DLBCL. However, improved dosing regimens and treatment methods are needed.

### SUMMARY

Provided herein are combination therapies for the treatment of cancer, and related methods and compositions. In some embodiments, combination therapies provided herein include a CD47 blocking agent and anti-CD20/anti-CD3 bispecific antibody. In some embodiments, the CD47 blocking agent is a SIRPa-Fc fusion protein (for example, TTI-622) and the anti-CD20/anti-CD3 bispecific antibody is glofitamab.

In some embodiments, provided herein is a method of treating a cancer in a patient, the method comprising administering a combination therapy comprising a CD47 blocking agent and an anti-CD20/anti-CD3 bispecific antibody to the patient.

In some embodiments, provided herein is a method of treating a cancer in a patient, the method comprising administering a combination therapy comprising a CD47 blocking agent and an anti-CD20/anti-CD3 bispecific antibody to the patient, wherein the CD47 blocking agent is a SIRPaFc fusion protein comprising the amino acid sequence of SEQ ID NO: 7 (TTI-622), and wherein the anti-CD20/anti-CD3 bispecific antibody is glofitamab. Optionally, the SIRPaFc fusion protein is maplirpacept.

In some embodiments, provided herein is a method of treating a cancer in a patient, the method comprising administering a combination therapy comprising a CD47 blocking agent and an anti-CD20/anti-CD3 bispecific antibody to the patient, wherein the CD47 blocking agent and the anti-CD20/anti-CD3 bispecific antibody are administered to the patient for at least a lead-in cycle and a first cycle, wherein the lead-in cycle precedes the first cycle, wherein each cycle is 21 days, wherein the anti-CD20/anti-CD3 bispecific antibody is administered on days 8 and 15 of the lead-in cycle, and on day 1 of the first cycle, wherein the method further comprises administering an anti-CD20 monospecific antibody on day 1 of the lead-in cycle, and wherein the CD47 blocking agent is administered on days 1, 8, and 15 of the first cycle.

In some embodiments, provided herein is a method of treating a cancer in a patient, the method comprising administering a combination therapy comprising a CD47 blocking agent and an anti-CD20/anti-CD3 bispecific antibody to the patient, wherein the CD47 blocking agent and the anti-CD20/anti-CD3 bispecific antibody are administered to the patient for at least a lead-in cycle, a first cycle, second cycle, third cycle and fourth cycle, wherein the lead-in cycle precedes the first cycle, wherein each cycle is 21 days, wherein the anti-CD20/anti-CD3 bispecific antibody is administered on days 8 and 15 of the lead-in cycle, and on day 1 of the first, second, third, and fourth cycle, wherein the method further comprises administering an anti-CD20 monospecific antibody on day 1 of the lead-in cycle, and wherein the CD47 blocking agent is administered on days 1, 8, and 15 of the first, second, and third cycle and on day 1 of the fourth cycle.

### BRIEF DESCRIPTION OF THE DRAWINGS

FIG. 1 depicts exemplary dosing regimens combining maplirpacept (TTI-622), glofitamab, and obinutuzumab. Each cycle is 21 days. Regimens are shown for cycle 0 (C0), cycles 1-3 (C1-3), cycles 4-11 (C4-11), and cycles 12 and beyond (C12+).

### DETAILED DESCRIPTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:

E1. A method of treating a cancer in a patient, the method comprising administering a combination therapy comprising a CD47 blocking agent and an anti-CD20/anti-CD3 bispecific antibody to the patient.

E2. The method of E1, wherein the CD47 blocking agent comprises a CD47-binding form of human SIRPalpha (SIRPa).

E3. The method of E2, wherein the CD47-binding form of human SIRPa is a CD47-binding fragment of human SIRPa.

E4. The method of E3, wherein the CD47-binding fragment of human SIRPa comprises the IgV domain of human SIRPa.

E5. The method of any one of E1-E4, wherein the CD47 blocking agent comprises an Fc fusion protein comprising the IgV domain of human SIRPa variant 2 attached to an antibody Fc region (SIRPaFc fusion protein).

E6. The method of E5, wherein the SIRPaFc fusion protein comprises a SIRPa polypeptide comprising the amino acid sequence of SEQ ID NO: 1.

E7. The method of any one of E5-E6, wherein the SIRPaFc fusion protein comprises a SIRPa polypeptide comprising the amino acid sequence of SEQ ID NO: 2.

E8. The method of any one of E5-E7, wherein the SIRPaFc fusion protein comprises the amino acid sequence of SEQ ID NO: 6 or SEQ ID NO: 7.

E9. The method of any one of E5-E8, wherein the SIRPaFc fusion protein comprises a SIRPa polypeptide comprising the amino acid sequence of SEQ ID NO: 1 or a variant thereof having one, two, three, four, or five amino acid substitutions as compared the sequence of SEQ ID NO: 1.

E10. The method of any one of E1-E9, wherein the anti-CD20/anti-CD3 bispecific antibody comprises a first antigen binding site that binds to CD3 and a second antigen binding site that binds to CD20, wherein the first antigen binding site comprises a VH and a VL, wherein the second antigen binding site comprises a VH and a VL, and wherein one or both of:
a) the first antigen binding site VH comprises the heavy chain CDR (HCDR) 1 of SEQ ID NO: 18, the HCDR2 of SEQ ID NO: 19, and the HCDR3 of SEQ ID NO: 20; and the first antigen binding site VL comprises the light chain CDR (LCDR) 1 of SEQ ID NO: 21, the LCDR2 of SEQ ID NO: 22, and the LCDR3 of SEQ ID NO: 23; and/or
b) the second antigen binding site VH comprises the heavy chain CDR (HCDR) 1 of SEQ ID NO: 10, the HCDR2 of SEQ ID NO: 11, and the HCDR3 of SEQ ID NO: 12; and the second antigen binding site VL comprises the light chain CDR (LCDR) 1 of SEQ ID NO: 13, the LCDR2 of SEQ ID NO: 14, and the LCDR3 of SEQ ID NO: 15.

E11. The method of E10, wherein the first antigen binding site VH comprises a sequence that is at least about 95%, 96%, 97%, 98%, 99%, or 100% identical to the amino acid sequence of SEQ ID NO: 24, the first antigen binding site VL comprises a sequence that is at least about 95%, 96%, 97%, 98%, 99%, or 100% identical to the amino acid sequence of SEQ ID NO: 25, the second antigen binding site VH comprises a sequence that is at least about 95%, 96%, 97%, 98%, 99%, or 100% identical to the amino acid sequence of SEQ ID NO: 16, and/or the second antigen binding site VL comprises a sequence that is at least about 95%, 96%, 97%, 98%, 99%, or 100% identical to the amino acid sequence of SEQ ID NO: 17.

E12. The method of any one of E1-E11, wherein the anti-CD20/anti-CD3 bispecific antibody comprises a polypeptide comprising the sequence of SEQ ID NO: 26, a polypeptide comprising the sequence of SEQ ID NO: 27, a polypeptide comprising the sequence of SEQ ID NO: 28, and a polypeptide comprising the sequence of SEQ ID NO: 29.

E13. The method of any one of E1-E12, wherein the anti-CD20/anti-CD3 bispecific antibody is glofitamab.

E14. The method of any one of E1-E13, wherein the CD47 blocking agent is an anti-CD47 or an anti-SIRPa antibody.

E15. The method of any one of E1-E14, wherein the CD47 blocking agent and the anti-CD20/anti-CD3 bispecific antibody are administered to the patient for at least a first cycle, second cycle, third cycle, and fourth cycle, wherein each cycle is 21 days, wherein the CD47 blocking agent is administered on days 1, 8, and 15 of the first, second, and third cycle and on day 1 of the fourth cycle.

E16. The method of any one of E1-E15, wherein the CD47 blocking agent and the anti-CD20/anti-CD3 bispecific antibody are administered to the patient for at least a first cycle, second cycle, third cycle, and fourth cycle, wherein each cycle is 21 days, wherein the anti-CD20/anti-CD3 bispecific antibody is administered on day 1 of the first, second, third cycle and fourth cycle.

E17. The method of any one of E1-E16, wherein the CD47 blocking agent and the anti-CD20/anti-CD3 bispecific antibody are administered to the patient for at least a first cycle, second cycle, third cycle, and fourth cycle, wherein each cycle is 21 days, wherein the CD47 blocking agent is administered on days 1, 8, and 15 of the first cycle, second cycle, and third cycle and on day 1 of the fourth cycle, and wherein the anti-CD20/anti-CD3 bispecific antibody is administered on day 1 of the first, second, third, and fourth cycle.

E18. The method of any one of E1-E17, wherein the CD47 blocking agent and the anti-CD20/anti-CD3 bispecific antibody are administered to the patient for at least a lead-in cycle, a first cycle, second cycle, third cycle and fourth cycle, wherein the lead-in cycle precedes the first cycle, wherein each cycle is 21 days, wherein the anti-CD20/anti-CD3 bispecific antibody is administered on days 8 and 15 of the lead-in cycle, on day 1 of the first, second, third, and fourth cycle.

E19. The method of E18, further comprising administering an anti-CD20 monospecific antibody on day 1 of the lead-in cycle.

E20. The method of any one of E1-E19, wherein the CD47 blocking agent and the anti-CD20/anti-CD3 bispecific antibody are administered to the patient for at least a first cycle, second cycle, third cycle, and fourth cycle, wherein each cycle is 21 days, wherein the CD47 blocking agent is administered at doses comprising 4 mg/kg, 8 mg/kg, 16 mg/kg, 300 mg, 600 mg, or 1200 mg on days 1, 8, and 15 of the first, second, and third cycle and at 10 mg/kg, 18 mg/kg, 28 mg/kg, 600 mg, 1200 mg, or 2400 mg on day 1 of the fourth cycle.

E21. The method of E20, wherein the CD47 blocking agent is administered at doses comprising 4 mg/kg or 300 mg on days 1, 8, and 15 of the first, second, and third cycle and at 10 mg/kg or 600 mg on day 1 of the fourth cycle, the CD47 blocking agent is administered at doses comprising 8 mg/kg or 600 mg on days 1, 8, and 15 of the first, second, and third cycle and 18 mg/kg or 1200 mg-on day 1 of the fourth cycle, or the CD47 blocking agent is administered at doses comprising 16 mg/kg or 1200 mg on days 1, 8, and 15 of the first, second, and third cycle and at 28 mg/kg or 2400 mg on day 1 of the fourth cycle, optionally wherein the CD47 blocking agent is administered at doses comprising 300 mg on days 1, 8, and 15 of the first, second, and third cycle and at 600 mg on day 1 of the fourth cycle, the CD47 blocking agent is administered at doses comprising 600 mg on days 1, 8, and 15 of the first, second, and third cycle and 1200 mg on day 1 of the fourth cycle, or the CD47 blocking agent is administered at doses comprising 1200 mg on days 1, 8, and 15 of the first, second, and third cycle and at 2400 mg on day 1 of the fourth cycle.

E22. The method of any one of E1-E21, wherein the CD47 blocking agent and the anti-CD20/anti-CD3 bispecific antibody are administered to the patient for at least a first cycle, second cycle, third cycle and fourth cycle, wherein each cycle is 21 days, wherein the anti-CD20/anti-CD3 bispecific antibody is administered at doses comprising 30 mg on day 1 of the first, second, third, and fourth cycle.

E23. The method of any one of E1-E22, wherein the CD47 blocking agent and the anti-CD20/anti-CD3 bispecific antibody are administered to the patient for at least a first cycle, second cycle, third cycle and fourth cycle, wherein each cycle is 21 days, wherein the CD47 blocking agent is administered at doses comprising 4 mg/kg, 8 mg/kg, 16 mg/kg, 300 mg, 600 mg, or 1200 mg on days 1, 8, and 15 of the first, second, and third cycle and at 10 mg/kg, 18 mg/kg, 28 mg/kg, 600 mg, 1200 mg, or 2400 mg on day 1 of the fourth cycle, and wherein the anti-CD20/anti-CD3 bispecific antibody is administered at doses comprising 30 mg on day 1 of the first, second, third, and fourth cycle, optionally wherein the CD47 blocking agent is administered at doses comprising 300 mg, 600 mg, or 1200 mg on days 1, 8, and 15 of the first, second, and third cycle and at 600 mg, 1200 mg, or 2400 mg on day 1 of the fourth cycle.

E24. The method of any one of E1-E23, wherein the CD47 blocking agent and the anti-CD20/anti-CD3 bispecific antibody are administered to the patient for at least a lead-in cycle, a first cycle, second cycle, third cycle, and fourth cycle, wherein the lead-in cycle precedes the first cycle, wherein each cycle is 21 days, wherein the anti-CD20/anti-CD3 bispecific antibody is administered at doses comprising 2.5 mg on day 8 and 10 mg on day 15 of the lead-in cycle, 30 mg on day 1 of the first, second, third, and fourth cycle.

E25. The method of E24 further comprising administering an anti-CD20 monospecific antibody on day 1 of the lead-in cycle.

E26. The method of E19 or E25, wherein the anti-CD20 monospecific antibody is administered at a dose of 1000 mg.

E27. The method of any one of E19, E25, or E26, wherein the anti-CD20 monospecific antibody is obinutuzumab.

E28. The method of any one of E15-E27, further comprising a fifth cycle and up to an eleventh cycle, wherein the CD47 blocking agent is administered at doses of 10 mg/kg, 18 mg/kg, 28 mg/kg, 600 mg, 1200 mg, or 2400 mg on day 1 of the fifth cycle through eleventh cycle, and the anti-CD20/anti-CD3 bispecific antibody is administered at doses comprising 30 mg on day 1 of the fifth cycle through eleventh cycle, optionally wherein the CD47 blocking agent is administered at doses of 600 mg, 1200 mg, or 2400 mg on day 1 of the fifth cycle through eleventh cycle.

E29. The method of E28, further wherein the CD47 blocking agent is administered on day 1 of a twelfth and additional cycles until disease progression.

E30. A method of treating a cancer in a patient, the method comprising administering a combination therapy comprising a CD47 blocking agent and an anti-CD20/anti-CD3 bispecific antibody to the patient, wherein the CD47 blocking agent is a SIRPaFc fusion protein comprising the amino acid sequence of SEQ ID NO: 7 (TTI-622), and wherein the anti-CD20/anti-CD3 bispecific antibody is glofitamab. Optionally, the SIRPaFc fusion protein is maplirpacept.

E31. The method of E30, further comprising administering an anti-CD20 monospecific antibody to the patient.

E32. The method of E31, wherein the anti-CD20 monospecific antibody is obinutuzumab.

E33. A method of treating a cancer in a patient, the method comprising administering a combination therapy comprising a CD47 blocking agent and an anti-CD20/anti-CD3 bispecific antibody to the patient, wherein the CD47 blocking agent and the anti-CD20/anti-CD3 bispecific antibody are administered to the patient for at least a lead-in cycle, a first cycle, second cycle, third cycle and fourth cycle, wherein the lead-in cycle precedes the first cycle, wherein each cycle is 21 days, wherein the anti-CD20/anti-CD3 bispecific antibody is administered on days 8 and 15 of the lead-in cycle, and on day 1 of the first, second, third, and fourth cycle, wherein the method further comprises administering an anti-CD20 monospecific antibody on day 1 of the lead-in cycle, and wherein the CD47 blocking agent is administered on days 1, 8, and 15 of the first, second, and third cycle and on day 1 of the fourth cycle.

E34. The method of E33, wherein the anti-CD20/anti-CD3 bispecific antibody is administered at doses comprising 2.5 mg on day 8 and 10 mg on day 15 of the lead-in cycle, and 30 mg on day 1 of the first, second, third, and fourth cycle, wherein the anti-CD20 monospecific antibody is administered at a dose of 1000 mg, and wherein the CD47 blocking agent is administered at i) doses comprising 4 mg/kg or 300 mg on days 1, 8, and 15 of the first, second, and third cycle and at 10 mg/kg or 600 mg on day 1 of the fourth cycle, ii) doses comprising 8 mg/kg or 600 mg on days 1, 8, and 15 of the first, second, and third cycle and 18 mg/kg or 1200 mg on day 1 of the fourth cycle, or iii) doses comprising 16 mg/kg or 1200 mg on days 1, 8, and 15 of the first, second, and third cycle and at 28 mg/kg or 2400 mg on day 1 of the fourth cycle, optionally wherein the CD47 blocking agent is administered at i) doses comprising 300 mg on days 1, 8, and 15 of the first, second, and third cycle and at 600 mg on day 1 of the fourth cycle, ii) doses comprising 600 mg on days 1, 8, and 15 of the first, second, and third cycle and 1200 mg on day 1 of the fourth cycle, or iii) doses comprising 1200 mg on days 1, 8, and 15 of the first, second, and third cycle and 2400 mg on day 1 of the fourth cycle.

E35. The method of E33 or E34, wherein the CD47 blocking agent is a SIRPaFc fusion protein comprising the amino acid sequence of SEQ ID NO: 7 (TTI-622), wherein the anti-CD20/anti-CD3 bispecific antibody is glofitamab, and wherein the anti-CD20 antibody is obinutuzumab.

E36. The method of any one of E1-E35 or E45-E57, wherein the cancer is a blood cancer or a solid tumor cancer.

E37. The method of any one of E1-E36 or E45-E57, wherein the cancer is selected from the group consisting of acute lymphocytic leukemia (ALL); acute myeloid leukemia (AML) and p53 mutated AML; chronic lymphocytic leukemia (CLL); chronic myelogenous leukemia (CML); myeloproliferative disorder/neoplasm (MPDS), diffuse large B cell lymphoma (DLBCL), myelodysplastic syndrome, lymphoma, T cell lymphoma, Hodgkin's lymphoma, indolent non-Hodgkin's lymphoma, aggressive non-Hodgkin's lymphoma, Burkitt's lymphoma, small cell follicular lymphoma, large cell follicular lymphoma. myeloma, multiple myeloma (MM), giant cell myeloma, heavy-chain myeloma, light chain or Bence-Jones myeloma, sarcoma, soft tissue sarcoma, leiomyosarcoma (LMS), undifferentiated pleomorphic sarcoma, myxofibrosarcoma, dedifferentiated liposarcoma, angiosarcoma, or epithelioid sarcoma, optionally wherein the cancer is relapsed or refractory.

E38. The method of any one of E1-E37 or E45-E57, wherein the cancer is relapsed or refractory (R/R) diffuse large B cell lymphoma (DLBCL).

E39. The method of any one of E1-E38 or E45-E57, wherein the patient has previously been treated with 1 to 3 lines of therapy.

E40. The method of any one of E1-E39 or E45-E57, wherein at least the CD47 blocking agent is administered until disease progression.

E41. The method of any one of claims E1-E40 or E45-E57, wherein the patient has CD47-positive cancer cells.

E42. A CD47 blocking agent or anti-CD20/anti-CD3 bispecific antibody for use in the treatment of cancer according to the method of any one of E1-E41 or E45-E57.

E43. Use of a CD47 blocking agent or an anti-CD20/anti-CD3 bispecific antibody in the manufacture of a medicament for the treatment of cancer according to the method of any one of E1-E41 or E45-E57.

E44. A kit comprising one or both of a CD47 blocking agent and an anti-CD20/anti-CD3 bispecific antibody and instructions for use according to the method of any one of E1-E41, and optionally further comprising one or more additional therapeutic agents according to the method of any one of E1-E41 or E45-E57.

E45. The method of any one of E1-E14, wherein the CD47 blocking agent and the anti-CD20/anti-CD3 bispecific antibody are administered to the patient for at least a first cycle, wherein each cycle is 21 days, wherein the CD47 blocking agent is administered on days 1, 8, and 15 of the first cycle.

E46. The method of any one of E1-E15 or E45, wherein the CD47 blocking agent and the anti-CD20/anti-CD3 bispecific antibody are administered to the patient for at least a first cycle, wherein each cycle is 21 days, wherein the anti-CD20/anti-CD3 bispecific antibody is administered on day 1 of the first cycle.

E47. The method of any one of E1-E16 or E45-E46, wherein the CD47 blocking agent and the anti-CD20/anti-CD3 bispecific antibody are administered to the patient for at least a first cycle, wherein each cycle is 21 days, wherein the CD47 blocking agent is administered on days 1, 8, and 15 of the first cycle, and wherein the anti-CD20/anti-CD3 bispecific antibody is administered on day 1 of the first cycle.

E48. The method of any one of E1-E17 or E45-E47, wherein the CD47 blocking agent and the anti-CD20/anti-CD3 bispecific antibody are administered to the patient for at least a lead-in cycle and a first cycle, wherein the lead-in cycle precedes the first cycle, wherein each cycle is 21 days, wherein the anti-CD20/anti-CD3 bispecific antibody is administered on days 8 and 15 of the lead-in cycle and on day 1 of the first cycle.

E49. The method of E48, further comprising administering an anti-CD20 monospecific antibody on day 1 of the lead-in cycle.

E50. The method of any one of E1-E19 or E45-E49, wherein the CD47 blocking agent and the anti-CD20/anti-CD3 bispecific antibody are administered to the patient for at least a first cycle, wherein each cycle is 21 days, wherein the CD47 blocking agent is administered at doses comprising 4 mg/kg, 8 mg/kg, 16 mg/kg, 300 mg, 600 mg, or 1200 mg on days 1, 8, and 15 of the first cycle.

E51. The method of E50, wherein the CD47 blocking agent is administered at doses comprising 4 mg/kg or 300 mg on days 1, 8, and 15 of the first cycle, the CD47 blocking agent is administered at doses comprising 8 mg/kg or 600 mg on days 1, 8, and 15 of the first cycle, or the CD47 blocking agent is administered at doses comprising 16 mg/kg or 1200 mg on days 1, 8, and 15 of the first cycle.

E52. The method of any one of E1-E21 or E45-E51, wherein the CD47 blocking agent and the anti-CD20/anti-CD3 bispecific antibody are administered to the patient for at least a first cycle, wherein each cycle is 21 days, wherein the anti-CD20/anti-CD3 bispecific antibody is administered at doses comprising 30 mg on day 1 of the first cycle.

E53. The method of any one of E1-E22 or E45-E52, wherein the CD47 blocking agent and the anti-CD20/anti-CD3 bispecific antibody are administered to the patient for at least a first cycle, wherein each cycle is 21 days, wherein the CD47 blocking agent is administered at doses comprising 4 mg/kg, 8 mg/kg, 16 mg/kg, 300 mg, 600 mg, or 1200 mg on days 1, 8, and 15 of the first cycle, and wherein the anti-CD20/anti-CD3 bispecific antibody is administered at doses comprising 30 mg on day 1 of the first cycle.

E54. The method of any one of E1-E23 or E45-E53, wherein the CD47 blocking agent and the anti-CD20/anti-CD3 bispecific antibody are administered to the patient for at least a lead-in cycle and a first cycle, wherein the lead-in cycle precedes the first cycle, wherein each cycle is 21 days, wherein the anti-CD20/anti-CD3 bispecific antibody is administered at doses comprising 2.5 mg on day 8 and 10 mg on day 15 of the lead-in cycle, and 30 mg on day 1 of the first cycle.

E55. The method of E54 further comprising administering an anti-CD20 monospecific antibody on day 1 of the lead-in cycle.

E56. The method of E49 or E55, wherein the anti-CD20 monospecific antibody is administered at a dose of 1000 mg.

E57. The method of any one of E49, E55, or E56, wherein the anti-CD20 monospecific antibody is obinutuzumab.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies. Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter (e.g., the dose of SIRPaFc fusion protein) means that the parameter may vary by as much as 10% below or above the stated numerical value for that parameter. For example, a dose of about 5 mg means 5%+10%, i.e. it may vary between 4.5 mg and 5.5 mg.

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder or condition is cancer.

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and (3) ameliorating the disease; for example, ameliorating a disease, condition or disorder in an individual that is experiencing or displaying the pathology or symptomatology of the disease, condition or disorder (i.e., reversing the pathology or symptomatology).

The term “CD47+” (or CD47+) is used with reference to the phenotype of cells targeted for binding by SIRPalpha fusion proteins or other CD47-binding agents. Cells that are CD47⁺ can be identified by flow cytometry using CD47 antibody as the affinity ligand. CD47 antibodies that are labeled appropriately are available commercially for this use (for example, the antibody product of clone B6H12 is available from Santa Cruz Biotechnology). The cells examined for CD47 phenotype can include standard tumour biopsy samples including particularly blood samples taken from the subject suspected of harbouring endogenous CD47+ cancer cells. CD47 disease cells of particular interest as targets for therapy with SIRPalpha fusion proteins are those that “over-express” CD47. These CD47+ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry as exemplified herein or by immunostaining or by gene expression analysis or the like, to be greater than the level measurable on a counterpart cell having a CD47 phenotype that is normal for that cell type.

A “bispecific antibody” refers to a molecule that has binding specificity for at least two different epitopes. In some embodiments, bispecific antibodies can bind simultaneously two different antigens. In other embodiments, the two different epitopes may reside on the same antigen. In certain embodiments, the bispecific antibody is capable of simultaneously binding two antigens expressed on two distinct cells.

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In some embodiments, CD3 is human CD3, particularly the epsilon subunit of human CD3 (CD38). The amino acid sequence of human CD38 is shown in UniProt accession no. P07766 (version 144), or NCBI RefSeq NP_000724.1. The amino acid sequence of cynomolgus [Macaca fascicularis] CD3E is shown in NCBI GenBank no. BAB71849.1.

“CD20”, also known as “B-lymphocyte antigen B1”, refers to any native CD20 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD20 as well as any form of CD20 that results from processing in the cell. The term also encompasses naturally occurring variants of CD20, e.g., splice variants or allelic variants. In some embodiments, CD20 is human CD20. Human CD20 is described in UniProt accession no. P11836 (entry version 200).

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196 (4): 901-917, 1987). As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)). Unless otherwise indicated, the CDRs are determined according to Kabat et al., supra. One of skill in the art will understand that the CDR designations can also be determined according to Chothia, supra, McCallum, supra, or any other scientifically accepted nomenclature system. “Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1 (L1)-FR2-H2 (L2)-FR3-H3 (L3)-FR4.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

Antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This particularly may be the case where the final two C-terminal amino acids of the heavy chain are glycine (G446) and lysine (K447, numbering according to Kabat EU index). Therefore, the C-terminal lysine, or the C-terminal glycine and lysine, of an antibody or SIRPaFc region provided herein may or may not be present.

### CD47 Blocking Agents

Dosing regimens and methods provided herein include a CD47 blocking agent. As used herein, a CD47 blocking agent can be any molecule that interferes with and dampens or blocks signal transmission that results when CD47 interacts with macrophage-presented SIRPa.

In some embodiments, a CD47-binding form of human SIRPa is the CD47 blocking agent for use in the regimens and methods provided herein. These molecules are based on the extracellular region of human SIRPa. They comprise at least a region of the extracellular region sufficient to confer effective CD47 binding affinity and specificity. So-called “soluble” forms of SIRPa, lacking the membrane anchoring component, are described in the literature and include those referenced in WO 2010/070047 (Novartis), WO2013/109752 (Stanford), and WO2014/094122 (Trillium), each incorporated by reference in its entirety.

In some embodiments, the soluble form of SIRPa is an Fc fusion. More particularly, the drug suitably comprises the human SIRPa protein, in a form fused directly, or indirectly, with an antibody constant region, or Fc (fragment crystallisable). Unless otherwise stated, the term “human SIRPa” as used herein refers to a wild type, endogenous, mature form of human SIRPa. In humans, the SIRPa protein is found in two major forms. One form, the variant 1 or V1 form, has the amino acid sequence set out as NCBI RefSeq NP_542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPa constitute about 80% of the forms of SIRPa present in humans, and both are embraced herein by the term “human SIRPa”. Also embraced by the term “human SIRPa” are the minor forms thereof that are endogenous to humans and have the same property of triggering signal transduction through CD47 upon binding thereto. The present invention is directed most particularly to the drug combinations that include the human SIRP variant 2 form, or V2.

In the dosing regimens and methods provided herein, useful SIRPaFc fusion proteins comprise one of the three so-called immunoglobulin (lg) domains that lie within the extracellular region of human SIRPa. More particularly, the present SIRPaFc proteins incorporate residues 32-137 of human SIRPa (a 106-mer), which constitute and define the IgV domain of the V2 form according to current nomenclature. This SIRPa sequence, shown below, is referenced herein as SEQ ID NO: 1.

[SEQ ID NO: 1]
EELQVIQPDKSVSVAAGESAILHCTVTSLIPVGPIQWFRGAGPARELIYN
QKEGHFPRVTTVSESTKRENMDFSISISNITPADAGTYYCVKFRKGSPDT
EFKSGA

In some embodiments, SIRPaFc fusion proteins incorporate the IgV domain as defined by SEQ ID NO: 1, and additional, flanking residues contiguous within the SIRPa sequence. This form of the IgV domain, represented by residues 31-148 of the V2 form of human SIRPa, is a 118-mer having SEQ ID NO: 2 shown below:

[SEQ ID NO: 2]
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPS

The present SIRPa fusion proteins can also incorporate an Fc region having effector function. Fc refers to “fragment crystallisable” and represents the constant region of an antibody comprised principally of the CH2 and CH3 domains of the heavy chain constant region and components within the hinge region. Suitable Fc components include those having effector function. An Fc component “having effector function” is an Fc component having at least some effector function, such as at least some contribution to antibody-dependent cellular cytotoxicity or some ability to fix complement. Also, the Fc will at least bind to Fc receptors. These properties can be revealed using assays established for this purpose. Functional assays include the standard chromium release assay that detects target cell lysis. By this definition, an Fc region that is wild type IgG1 or IgG4 has effector function, whereas the Fc region of a human IgG4 mutated to eliminate effector function, such as by incorporation of an alteration series that includes Pro233, Val234, Ala235 and deletion of Gly236 (EU), is considered not to have effector function. In some embodiments, the Fc is based on human antibodies of the IgG1 isotype. The Fc region of these antibodies will be readily identifiable to those skilled in the art. In some embodiments, the Fc region includes the lower hinge-CH2-CH3 domains.

In a specific embodiment, the Fc region is based on the amino acid sequence of a human IgG1 set out as P01857 in UniProtKB/Swiss-Prot, residues 104-330, and has the amino acid sequence shown below and referenced herein as SEQ ID NO: 3:

[SEQ ID NO: 3]
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK

Thus, in some embodiments, the Fc region has either a wild type or consensus sequence of an IgG1 constant region. In alternative embodiments, the Fc region incorporated in the fusion protein is derived from any IgG1 antibody having a typical effector-active constant region. The sequences of such Fc regions can correspond, for example, with the Fc regions of any of the following IgG1 sequences (all referenced from GenBank), for example: BAG65283 (residues 242-473), BAC04226.1 (residues 247-478), BAC05014.1 (residues 240-471), CAC20454.1 (residues 99-320), BAC05016.1 (residues 238-469), BAC85350.1 (residues 243-474), BAC85529.1 (residues 244-475), and BAC85429.1 (residues (238-469).

In other embodiments, the Fc region has a sequence of a wild type human IgG4 constant region. In alternative embodiments, the Fc region incorporated in the fusion protein is derived from any IgG4 antibody having a constant region with effector activity that is present but, naturally, is significantly less potent than the IgG1 Fc region. The sequences of such Fc regions can correspond, for example, with the Fc regions of any of the following IgG4 sequences: P01861 (residues 99-327) from UniProtKB/Swiss-Prot and CAC20457.1 (residues 99-327) from GenBank.

In some embodiments, the Fc region is based on the amino acid sequence of a human IgG4 set out as P01861 in UniProtKB/Swiss-Prot, residues 99-327, and has the amino acid sequence shown below and referenced herein as SEQ ID NO: 4:

[SEQ ID NO: 4]
ESKYGPPCPSCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNV
FSCSVMHEALHNHYTQKSLSLSLGK

In some embodiments, the Fc region incorporates one or more alterations, usually not more than about 10, e.g., up to 1, 2, 3, 4, 5 or 6 such alterations, including amino acid substitutions that affect certain Fc properties. In one specific embodiment, the Fc region incorporates an alteration at position 228 (EU numbering), in which the serine at this position is substituted by a proline (S²²⁸P), thereby to stabilize the disulfide linkage within the Fc dimer. Other alterations within the Fc region can include substitutions that alter glycosylation, such as substitution of Asn²⁹⁷ by glycine or alanine; half-life enhancing alterations such as T²⁵²L, T²⁵³S, and T²⁵⁶F as taught in U.S. Pat. No. 62,777,375, and many others. Particularly useful are those alterations that enhance Fc properties while remaining silent with respect to conformation, e.g., retaining Fc receptor binding. In another embodiment, the Fc region is modified to increase its biological half-life. Various approaches are possible. For example, one or more of the following mutations can be introduced; T252L, T254S, T256F, as described in U.S. Pat. No. 6,277,375.

In a specific embodiment, and in the case where the Fc component is an IgG4 Fc, the Fc incorporates at least the S²²⁸P mutation, and has the amino acid sequence set out below and referenced herein as SEQ ID NO: 5:

[SEQ ID NO: 5]
ESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNV
FSCSVMHEALHNHYTQKSLSLSLGK

The CD47 blocking agent used in the regimens and methods provided herein is thus in some embodiments a SIRP fusion protein useful to inhibit the binding of human SIRPa and human CD47, thereby to inhibit or reduce transmission of the signal mediated via SIRPa-bound CD47, the fusion protein comprising a human SIRPa component and, fused therewith, an Fc component, wherein the SIRPa component comprises or consists of a single IgV domain of human SIRPa V2 and the Fc component contains a human IgG Fc domain having effector function.

In one embodiment, the fusion protein comprises a SIRPa component comprising at least of residues 32-137 of the V2 form of wild type human SIRPa, i.e., SEQ ID NO: 1. In a preferred embodiment, the SIRPa component comprises residues 31-148 of the V2 form of human SIRPa, i.e., SEQ ID NO: 2. In one embodiment, the Fc component is the Fc component of the human IgG1 designated P01857, and in a specific embodiment has the amino acid sequence that incorporates the lower hinge-CH2-CH3 region thereof i.e., SEQ ID NO: 3. In another embodiment, the Fc component is the Fc component of the human IgG4 designated P01861, and in a specific embodiment has the amino acid sequence that incorporates the lower hinge-CH2-CH3 region thereof and the mutation S228P, i.e., SEQ ID NO: 5.

In some embodiments, the SIRPaFc fusion protein is provided and used in a secreted dimeric fusion form, wherein the fusion protein incorporates a SIRPa component having SEQ ID NO: 1 and preferably SEQ ID NO: 2 and, fused therewith, an Fc region having effector function and having SEQ ID NO: 3. When the SIRPa component is SEQ ID NO: 2 and the Fc region is SEQ ID NO: 3, the fusion protein comprises SEQ ID NO: 6, shown below:

[SEQ ID NO: 6]
EEELQVIQPDKSVSVAAGESAILHCTVTSLIPVGPIQWFRGAGPARELIYN
QKEGHFPRVTTVSESTKRENMDFSISISNITPADAGTYYCVKFRKGSPDTE
FKSGAGTELSVRAKPSDKTHTCPPCPAPELLGGPSVFLFPPKPKDTLMISR
TPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYS
KLTVDKSRWQQGNVFSCSVMHEALHNHYTQKSLSLSPGK

The SIRPaFc fusion protein of SEQ ID NO: 6 is also known as TTI-621 or ontorpacept. TTI-621/ontorpacept comprises a dimer of proteins of SEQ ID NO: 6.

In alternative embodiments, the Fc component of the fusion protein is based on an IgG4, and preferably an IgG4 that incorporates the S²²⁸P mutation. In the case where the fusion protein incorporates the preferred SIRPa IgV domain of SEQ ID NO: 2, and the IgG4 Fc region is SEQ ID NO: 5, the fusion protein comprises SEQ ID NO: 7, shown below:

[SEQ ID NO: 7]
EEELQVIQPDKSVSVAAGESAILHCTVTSLIPVGPIQWFRGAGPARELIYN
QKEGHFPRVTTVSESTKRENMDFSISISNITPADAGTYYCVKFRKGSPDTE
FKSGAGTELSVRAKPSESKYGPPCPPCPAPEFLGGPSVFLFPPKPKDTLMI
SRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFL
YSRLTVDKSRWQEGNVFSCSVMHEALHNHYTQKSLSLSLGK

The SIRPaFc fusion protein of SEQ ID NO: 7 is also known as TTI-622 and maplirpacept (recommended INN: 2631667-06-4, WHO Drug Information, Vol. 36, No. 2, 2022, List 127, p. 424-425). TTI-622/maplirpacept comprises a dimer of proteins of SEQ ID NO: 7.

In one embodiment of a dosing regimen or method provided herein, a SIRPaFc fusion protein comprises, as the SIRPa component of the fusion protein, a sequence that comprises the polypeptide of SEQ ID NO: 2. In one embodiment, the SIRPaFc fusion protein comprises the polypeptide of SEQ ID NO: 6 or SEQ ID NO: 7.

The SIRPα sequence incorporated within the SIRPaFc fusion protein can be varied, as described in the literature. This can eliminate glycosylation sites in the protein, such as at position 89 and elsewhere. Other, useful substitutions within SIRPa include one or more of the following: L4V/I, V6I/L, A21V, V27I/L, 131T/S/F, E47V/L, K53R, E54Q, H56P/R, S66T/G, K68R, V92I, F94V/L, V63I, and/or F103V.

In a SIRPaFc fusion protein, the SIRPa component and the Fc component are fused, either directly or indirectly, to provide a single chain polypeptide that may optionally be ultimately produced as a dimer in which the single chain polypeptides are coupled through inter-chain disulfide bonds formed within the Fc region. The nature of the fusing region is not critical. The fusion may be direct between the two components, with the SIRP component constituting the N-terminal end of the fusion and the Fc component constituting the C-terminal end. Alternatively, the fusion may be indirect, through a linker comprised of one or more amino acids, desirably genetically encoded amino acids, such as two, three, four, five, six, seven, eight, nine or ten amino acids, or any number of amino acids between 5 and 100 amino acids, such as between 5 and 50, 5 and 30 or 5 and 20 amino acids. A linker may comprise a peptide that is encoded by DNA constituting a restriction site, such as a BamHI, ClaI, EcoRI, HindIII, PstI, SalI and XhoI site and the like.

The linker amino acids typically and desirably have some flexibility to allow the Fc and the SIRP components to adopt their active conformations. Residues that allow for such flexibility typically are Gly, Asn and Ser, so that virtually any combination of these residues (and particularly Gly and Ser) within a linker is likely to provide the desired linking effect. In one example, such a linker is based on the so-called G4S sequence (Gly-Gly-Gly-Gly-Ser [SEQ ID NO: 8]) which may repeat as (G4S)n where n is 1, 2, 3 or more, or is based on (Gly)n, (Ser)n, (Ser-Gly)n or (Gly-Ser)n and the like. In another embodiment, the linker is GTELSVRAKPS [SEQ ID NO: 9]. This sequence constitutes SIRPa sequence that C-terminally flanks the IgV domain (it being understood that this flanking sequence could be considered either a linker or a different form of the IgV domain when coupled with the IgV minimal sequence described above). It is necessary only that the fusing region or linker permits the components to adopt their active conformations, and this can be achieved by any form of linker useful in the art.

In some embodiments, a SIRPaFc fusion protein (e.g. TTI-622) may administered in methods and regimens provided herein the range of 0.1 to 50 mg/kg subject body weight. For example, a SIRPaFc fusion protein dosage can be 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, or 50 mg/kg. SIRPaFc fusion protein dosages can also include, for example 2-40 mg/kg, 4-40 mg/kg, 5-50 mg/kg, 8-50 mg/kg, 8-40 mg/kg, 8-30 mg/kg, 8-28 mg/kg 10-50 mg/kg, 10-40 mg/kg, 10-30 mg/kg, 10-25 or 10-20 mg/kg. These dosages of SIRPaFc fusion protein can be administered to a subject, for example, once a week (Q1W), once every two weeks (Q2W), once every three weeks (Q3W), once every four weeks (Q4W), two times a month, once a month, once every two months, or once every three months. These dosing frequencies may be part of dosing cycles, such as 14-day, 21-day, or 28-day cycles.

In some embodiments, a SIRPaFc fusion protein provided herein [e.g. TTI-622 (SEQ ID NO: 8)] is administered as a “flat” (also referred to as a “fixed”) dose—i.e. the dose is the amount per patient, and the dose does not depend on the mass of the patient. In some embodiments, a SIRPaFc fusion protein such as TTI-622 is administered at a fixed dose of 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, or 3600 mg. A fixed dose of SIRPaFc fusion protein may be administered in various regimens. In some embodiments, the dose is administered to a patient weekly (QW), every 2 weeks (Q2W), every 3 weeks (Q3W), or every 4 weeks (Q4W).

In some embodiments, a SIRPaFc fusion protein is administered at a dose between a) a lower level of 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, or 2200 mg and b) an upper level of 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, or 3600 mg, wherein the lower level is a smaller value than the upper level.

Still other types of CD47 blockade agent can be used in the present methods and combinations, instead of or in addition to the SIRPa-based drugs. These other drugs include particularly anti-CD47 antibodies, which bind to CD47 and antagonize the interaction with SIRPa. By blocking that interaction, and because of the Fc region of the antibody, the effect of the CD47 antibodies can be similar to the effect of the SIRPa-based Fc fusion drugs. Examples of CD47 antibodies are described in the literature such as US2008/0107654 (Chugai), WO2009/091601 (Stanford), WO2013/119714 (InhibRx), WO2016/109415 (Celgene) and WO2016/081423 (Janssen). Because these antibodies bind red blood cells, a dosing regimen that takes this into account has been developed and is described in WO2014/149477. The properties of a useful anti-CD47 antibody include the ability to bind to CD47 in a way that ultimately inhibits signaling by SIRPa, i.e., as an antagonist. In some other embodiments, anti-SIRPa antibodies may also be used as the CD47 blocking agent.

### Anti-CD20/Anti-CD3 Bispecific Antibody

Dosing regimens and methods provided herein include an anti-CD20/anti-CD3 bispecific antibody. As used herein, an anti-CD20/anti-CD3 bispecific antibody can be any molecule that can simultaneously bind to both CD20 (e.g. on B cells) and CD3 (e.g. on T cells). Exemplary anti-CD20/anti-CD3 bispecific antibodies include glofitamab (Roche), mosunetuzumab (Roche), odronextamab (Regeneron), epcoritamab (Genmab/Abbvie), plamotamab (Xencor), and IGM-2323 (IGM Biosciences).

In some embodiments, the anti-CD20/anti-CD3 bispecific antibody is capable of crosslinking a CD3-expressing T cell and CD20-expressing target cell by simultaneous binding to CD3 and CD20. In even more preferred embodiments, such simultaneous binding results in lysis of the CD20-expressing target cell, particularly a CD20-expressing tumor cell. In some embodiments, such simultaneous binding results in activation of the T cell. In some embodiments, such simultaneous binding results in a cellular response of the T cell, selected from the group of: proliferation, differentiation, cytokine secretion, cytotoxic effector molecule release, cytotoxic activity, and expression of activation markers. In some embodiments, binding of the bispecific antibody to CD3 without simultaneous binding to the target cell CD20 does not result in T cell activation. In some embodiments, the T cell bispecific antibody is capable of re-directing cytotoxic activity of a T cell to a CD20-expressing target cell. In some embodiments, said re-direction is independent of MHC-mediated peptide antigen presentation by the CD20-expressing target cell and and/or specificity of the T cell.

In some embodiments, the anti-CD20/anti-CD3 bispecific antibody provides monovalent binding to each of CD20 and CD3. Thus, in some embodiments, the anti-CD20/anti-CD3 bispecific antibody comprises a first antigen binding site that binds to CD3, and a second antigen binding site that binds to CD20.

In some embodiments, the anti-CD20/anti-CD3 bispecific antibody comprises a single antigen binding site that binds to CD3, and two antigen binding sites that bind to CD20. Thus, in some embodiments, the anti-CD20/anti-CD3 bispecific antibody comprises a third antigen binding site, particularly a Fab molecule, more particularly a conventional Fab molecule, that binds to CD20. The third antigen binding site may incorporate, singly or in combination, all of the features of the second antigen binding site (e.g. the CDR sequences, variable region sequences, and/or amino acid substitutions in the constant regions). In some embodiments, the third antigen binding site is identical to the second antigen binding site (e.g. is also a conventional Fab molecule and comprises the same amino acid sequences). In other words, in some embodiments, the two anti-CD20 antigen binding sites in the molecule have the same sequences.

In some embodiments, an anti-CD20/anti-CD3 bispecific antibody for use in the methods and regimens provided herein is described e.g. in PCT publication no. WO 2016/020309 (incorporated herein by reference in its entirety).

In some embodiments, an anti-CD20/anti-CD3 bispecific antibody for use in the methods and regimens provided herein comprises a first antigen binding site that binds to CD3 and a second antigen binding site that binds to CD20, the first antigen binding site comprises a VH and a VL, and the first antigen binding site VH comprises the heavy chain CDR (HCDR) 1 of SEQ ID NO: 18, the HCDR2 of SEQ ID NO: 19, and the HCDR3 of SEQ ID NO: 20; and the first antigen binding site VL comprises the light chain CDR (LCDR) 1 of SEQ ID NO: 21, the LCDR2 of SEQ ID NO: 22, and the LCDR3 of SEQ ID NO: 23.

In some embodiments, an anti-CD20/anti-CD3 bispecific antibody for use in the methods and regimens provided herein comprises a first antigen binding site that binds to CD3 and a second antigen binding site that binds to CD20, the second antigen binding site comprises a VH and a VL, and the second antigen binding site VH comprises the heavy chain CDR (HCDR) 1 of SEQ ID NO: 10, the HCDR2 of SEQ ID NO: 11, and the HCDR3 of SEQ ID NO: 12; and the second antigen binding site VL comprises the light chain CDR (LCDR) 1 of SEQ ID NO: 13, the LCDR2 of SEQ ID NO: 14, and the LCDR3 of SEQ ID NO: 15.

In some embodiments, an anti-CD20/anti-CD3 bispecific antibody provided herein comprises a first antigen binding site that binds to CD3 and a second antigen binding site that binds to CD20, the first antigen binding site comprises a VH and a VL, and the second antigen binding site comprises a VH and a VL, the first antigen binding site VH comprises the heavy chain CDR (HCDR) 1 of SEQ ID NO: 18, the HCDR2 of SEQ ID NO: 19, and the HCDR3 of SEQ ID NO: 20; and the first antigen binding site VL comprises the light chain CDR (LCDR) 1 of SEQ ID NO: 21, the LCDR2 of SEQ ID NO: 22, and the LCDR3 of SEQ ID NO: 23, and the second antigen binding site VH comprises the heavy chain CDR (HCDR) 1 of SEQ ID NO: 10, the HCDR2 of SEQ ID NO: 11, and the HCDR3 of SEQ ID NO: 12; and the second antigen binding site VL comprises the light chain CDR (LCDR) 1 of SEQ ID NO: 13, the LCDR2 of SEQ ID NO: 14, and the LCDR3 of SEQ ID NO: 15.

In some embodiments, an anti-CD20/anti-CD3 bispecific antibody provided herein comprises a first antigen binding site that binds to CD3 and a second antigen binding site that binds to CD20, the first antigen binding site comprises a VH and a VL, and the second antigen binding site comprises a VH and a VL, the first antigen binding site comprises a VH comprising the amino acid sequence of SEQ ID NO: 24 and a VL comprising the amino acid sequence of SEQ ID NO: 25. In some embodiments, the first antigen binding site comprises a VH comprising a sequence that is at least about 95%, 96%, 97%, 98%, 99%, or 100% identical to the amino acid sequence of SEQ ID NO: 24 and a VL sequence that is at least about 95%, 96%, 97%, 98%, 99%, or 100% identical to the amino acid sequence comprising the amino acid sequence of SEQ ID NO: 25.

In some embodiments, an anti-CD20/anti-CD3 bispecific antibody provided herein comprises a first antigen binding site that binds to CD3 and a second antigen binding site that binds to CD20, the first antigen binding site comprises a VH and a VL, and the second antigen binding site comprises a VH and a VL, the second antigen binding site comprises a VH comprising the amino acid sequence of SEQ ID NO: 16 and a VL comprising the amino acid sequence of SEQ ID NO: 17. In some embodiments, the second antigen binding site comprises a VH comprising a sequence that is at least about 95%, 96%, 97%, 98%, 99%, or 100% identical to the amino acid sequence of SEQ ID NO: 16 and a VL sequence that is at least about 95%, 96%, 97%, 98%, 99%, or 100% identical to the amino acid sequence comprising the amino acid sequence of SEQ ID NO: 17.

In some embodiments, the anti-CD20/anti-CD3 bispecific antibody comprises
(i) a first antigen binding site that binds to CD3, comprising a heavy chain variable region (VH) comprising the heavy chain CDR (HCDR) 1 of SEQ ID NO: 18, the HCDR2 of SEQ ID NO: 19, and the HCDR3 of SEQ ID NO: 20; and a light chain variable region (VL) comprising the light chain CDR (LCDR) 1 of SEQ ID NO: 21, the LCDR2 of SEQ ID NO: 22, and the LCDR3 of SEQ ID NO: 23, wherein the first antigen binding site is a crossover Fab molecule wherein either the variable or the constant regions, particularly the variable regions, of the Fab light chain and the Fab heavy chain are exchanged;
(ii) a second and a third antigen binding site that bind to CD20, comprising a heavy chain variable region comprising the heavy chain CDR (HCDR) 1 of SEQ ID NO: 10, the HCDR2 of SEQ ID NO: 11, and the HCDR3 of SEQ ID NO: 12; and a light chain variable region comprising the light chain CDR (LCDR) 1 of SEQ ID NO: 13, the LCDR2 of SEQ ID NO: 14, and the LCDR3 of SEQ ID NO: 15, wherein the second and third antigen binding site are each a Fab molecule, particularly a conventional Fab molecule;
(iii) an Fc domain composed of a first and a second subunit,
wherein the second antigen binding site is fused at the C-terminus of the Fab heavy chain to the N-terminus of the Fab heavy chain of the first antigen binding site, and the first antigen binding site is fused at the C-terminus of the Fab heavy chain to the N-terminus of the first subunit of the Fc domain, and wherein the third antigen binding site is fused at the C-terminus of the Fab heavy chain to the N-terminus of the second subunit of the Fc domain.

In some embodiments, the first antigen binding site of the anti-CD20/anti-CD3 bispecific antibody is a crossover Fab molecule wherein the variable regions of the Fab light chain and the Fab heavy chain are exchanged, and the second and (where present) third antigen binding site of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

Particularly, in the above embodiments, in the constant domain CL of the second and the third Fab molecule under (ii) the amino acid at position 124 may be substituted by lysine (K) (numbering according to Kabat) and the amino acid at position 123 may be substituted by lysine (K) or arginine (R), particularly by arginine (R) (numbering according to Kabat), and in the constant domain CH1 of the second and the third Fab molecule under (ii) the amino acid at position 147 may be substituted by glutamic acid (E) (numbering according to Kabat EU index) and the amino acid at position 213 may be substituted by glutamic acid (E) (numbering according to Kabat EU index).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

In some embodiments, the first antigen binding site (binding to CD3) of the anti-CD20/anti-CD3 bispecific antibody comprises a heavy chain variable region sequence that is at least about 95%, 96%, 97%, 98%, 99% or 100% identical to the amino acid sequence of SEQ ID NO: 24 and a light chain variable region sequence that is at least about 95%, 96%, 97%, 98%, 99% or 100% identical to the amino acid sequence of SEQ ID NO: 25. In some embodiments, the first antigen binding site comprises the heavy chain variable region sequence of SEQ ID NO: 24 and the light chain variable region sequence of SEQ ID NO: 25.

In some embodiments, the second and (where present) third antigen binding site (binding to CD20) of the anti-CD20/anti-CD3 bispecific antibody comprise a heavy chain variable region sequence that is at least about 95%, 96%, 97%, 98%, 99% or 100% identical to the amino acid sequence of SEQ ID NO: 16 and a light chain variable region sequence that is at least about 95%, 96%, 97%, 98%, 99% or 100% identical to the amino acid sequence of SEQ ID NO: 17. In some embodiments, the second and (where present) third antigen binding site comprise the heavy chain variable region of SEQ ID NO: 16 and the light chain variable region of SEQ ID NO: 17.

In some embodiments, the Fc domain of the anti-CD20/anti-CD3 bispecific antibody is an IgG Fc domain. In particular embodiments, the Fc domain is an IgG1 Fc domain. In further particular embodiments, the Fc domain is a human Fc domain. In particularly preferred embodiments, the Fc domain is a human IgG1 Fc domain.

In some embodiments, the Fc domain of the anti-CD20/anti-CD3 bispecific antibody comprises a modification promoting the association of the first and the second subunit of the Fc domain. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

In specific embodiments, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine(S), threonine (T), and valine (V). The protuberance and cavity can be made by altering the nucleic acid encoding the polypeptides, e.g. by site-specific mutagenesis, or by peptide synthesis.

In specific such embodiments, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In further embodiments, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In preferred embodiments, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some embodiments, the Fc domain of the anti-CD20/anti-CD3 bispecific antibody comprises one or more amino acid substitution that reduces binding to an Fc receptor and/or effector function. In particular embodiments, the Fc receptor is an Fcγ receptor. In some embodiments, the Fc receptor is a human Fc receptor. In some embodiments, the Fc receptor is an activating Fc receptor. In specific embodiments, the Fc receptor is an activating human Fcγ receptor, more specifically human FcγRIIIa, FcγRI or FcγRIIa, most specifically human FcγRIIIa. In some embodiments, the effector function is one or more selected from the group of complement dependent cytotoxicity (CDC), antibody-dependent cell-mediated cytotoxicity (ADCC), antibody-dependent cellular phagocytosis (ADCP), and cytokine secretion. In particular embodiments, the effector function is ADCC.

Typically, the same one or more amino acid substitution is present in each of the two subunits of the Fc domain. In some embodiments, the Fc domain comprises an amino acid substitution at a position selected from the group of E233, L234, L235, N297, P331 and P329 (numberings according to Kabat EU index). In more specific embodiments, the Fc domain comprises an amino acid substitution at a position selected from the group of L234, L235 and P329 (numberings according to Kabat EU index). In particular embodiments, the Fc domain comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”). Specifically, in preferred embodiments, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In some such embodiments, the Fc domain is an IgG₁ Fc domain, particularly a human IgG₁ Fc domain.

In some embodiments, the antigen binding sites and the Fc region are fused to each other by peptide linkers, particularly by peptide linkers as included in the polypeptide of SEQ ID NO: 27 and the polypeptide of SEQ ID NO: 29. By “fused” is meant that the components (e.g. a Fab molecule and an Fc domain subunit) are linked by peptide bonds, either directly or via one or more peptide linkers.

In some embodiments, the anti-CD20/anti-CD3 bispecific antibody comprises a polypeptide (particularly two polypeptides) comprising a sequence that is at least 80%, 85%, 90%, 95%, 96%, 97%, 98%, or 99% identical to the sequence of SEQ ID NO: 26, a polypeptide comprising a sequence that is at least 80%, 85%, 90%, 95%, 96%, 97%, 98%, or 99% identical to the sequence of SEQ ID NO: 27, a polypeptide comprising a sequence that is at least 80%, 85%, 90%, 95%, 96%, 97%, 98%, or 99% identical to the sequence of SEQ ID NO: 28, and a polypeptide comprising a sequence that is at least 80%, 85%, 90%, 95%, 96%, 97%, 98%, or 99% identical to the sequence of SEQ ID NO: 29. In some embodiments, the anti-CD20/anti-CD3 bispecific antibody comprises a polypeptide (particularly two polypeptides) comprising the sequence of SEQ ID NO: 26, a polypeptide comprising the sequence of SEQ ID NO: 27, a polypeptide comprising the sequence of SEQ ID NO: 28, and a polypeptide comprising the sequence of SEQ ID NO: 29.

In some embodiments, the anti-CD20/anti-CD3 bispecific antibody is glofitamab (WHO Drug Information (International Nonproprietary Names for Pharmaceutical Substances), Recommended INN: List 83, 2020, vol. 34, no. 1, p. 39). Glofitamab comprises one CD3-binding site and two CD20-binding sites, and comprises the polypeptides of SEQ ID NOs: 26, 27, 28, and 29. The heavy chain polypeptides (SEQ ID NOs 27, 29) may further comprise the C-terminal lysine, or the C-terminal glycine and lysine, as indicated above.

### Anti-CD20 Monospecific Antibody

In some embodiments, dosing regimens and methods provided herein may include an anti-CD20 monospecific antibody. As used herein, an anti-CD20 monospecific antibody can be any molecule that specifically binds to only CD20 (and not also another antigen, such as CD3). Exemplary CD20 monospecific antibodies include obinutuzumab, rituximab, ofatumumab, ocrelizumab, and veltuzumab.

In some embodiments, a CD20 monospecific antibody may be administered prior to an anti-CD20/anti-CD3 bispecific antibody in a regimen or method provided herein, in order to reduce the risk of cytokine-release syndrome (CRS) by depleting peripheral and tissue based B cells prior to the administration of the anti-CD20/anti-CD3 bispecific antibody.

In a preferred embodiment, the anti-CD20 monospecific antibody for use in the dosing regimens and methods provided herein is obinutuzumab (CAS Number 949142-50-1; recommended INN, WHO Drug Information, Vol. 26, No. 4, 2012, p. 453). As used herein, obinutuzumab is synonymous for GA101. The tradename is GAZYVA® or GAZYVARO®. This replaces all previous versions (e.g. Vol. 25, No. 1, 2011, p. 75-76), and is formerly known as afutuzumab (recommended INN, WHO Drug Information, Vol. 23, No. 2, 2009, p. 176; Vol. 22, No. 2, 2008, p. 124).

### CD47 Blocking Agent and Anti-CD20/Anti-CD3 Bispecific Antibody Combination Therapies

Provided herein are combination therapies containing a CD47 blocking agent and an anti-CD20/anti-CD3 bispecific antibody. In some embodiments, the CD47 blocking agent is a SIRPa-Fc fusion protein (for example, TTI-622/maplirpacept) and the anti-CD20/anti-CD3 bispecific antibody is glofitamab.

Without being bound by theory, it is hypothesized that a combination of a CD47 blocking agent (e.g. TTI-622) with anti-CD20/anti-CD3 bispecific antibodies might result in enhanced efficacy due to their non-redundant and distinct mechanisms of action. By targeting CD47, an agent such as TTI-622 promotes tumor clearance by antibody-dependent cellular phagocytosis (ADCP); whereas an anti-CD20/anti-CD3 bispecific antibody such as glofitamab promotes tumor clearance by activating T cell mediated cytotoxicity. In addition, both TTI-622 and anti-CD20/anti-CD3 antibodies induce secretion of pro-inflammatory cytokines and chemokines, thereby facilitating the recruitment and activation of T cells and macrophages.

In some embodiments, a combination therapy containing a CD47 blocking agent and an anti-CD20/anti-CD3 bispecific antibody as provided herein may further include an anti-CD20 monospecific antibody. The anti-CD20 monospecific antibody may be administered prior to the anti-CD20/anti-CD3 bispecific antibody in order to reduce the risk of cytokine-release syndrome (CRS) triggered by the anti-CD20/anti-CD3 bispecific antibody.

In some embodiments, a combination therapy containing a CD47 blocking agent, an anti-CD20/anti-CD3 bispecific antibody, and an anti-CD20 bispecific antibody may include any of the dosage amounts and frequencies as described in Example 1.

SIRPaFc proteins provided herein display negligible binding to red blood cells. There is accordingly no need to account for an RBC “sink” when dosing with SIRPaFc fusion proteins provided herein. Relative to other CD47 blockade drugs that are bound by RBCs, it is estimated that the present SIRPaFc fusions can be effective at doses that are less than half the doses required for drugs that become RBC-bound, such as CD47 antibodies. Moreover, the SIRPaFc fusion proteins provided herein are a dedicated antagonist of the SIRPa-mediated signal, they displays negligible CD47 agonism when binding thereto. There is accordingly no need, when establishing medically useful unit dosing regimens, to account for any stimulation induced by the drug.

Dosing regimens and methods provided herein may be is useful to treat a variety of cancers. These include particularly CD47+ cancer cells and/or CD20+ cancer cells, including liquid (hematological) tumours. In one embodiment, dosing regimens and methods provided herein can used to inhibit the growth or proliferation of hematological cancers. As used herein, “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome. “Lymphoma” may refer to a Hodgkin's lymphoma, both indolent and aggressive non-Hodgkin's lymphoma, Burkitt's lymphoma, and follicular lymphoma (small cell and large cell), among others. Myeloma may refer to multiple myeloma (MM), giant cell myeloma, heavy-chain myeloma, and light chain or Bence-Jones myeloma. In particular embodiments, dosing regimens and methods provided herein are useful to treat T cell lymphomas that are a very heterogeneous group of lymphoid malignancies divided into cutaneous and peripheral TCL, which themselves are divided into nodal or extranodal types. CTCL derive from skin-homing T cells and consist of mycosis fungoides, Sezary syndrome, primary cutaneous T cell lymphoproliferative disorders, and anaplastic large cell lymphoma. The common features of TCL are aggressive course and poor response to therapy, with the exception of ALK and ALCL.

In some other embodiments, the hematological cancer treated with dosing regimens and methods is a CD47⁺ leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

In other embodiments, the hematological cancer treated with a dosing regimen or method provided herein is a CD47⁺ lymphoma or myeloma selected from Hodgkin's lymphoma, both indolent and aggressive non-Hodgkin's lymphoma, Burkitt's lymphoma, follicular lymphoma (small cell and large cell), multiple myeloma (MM), giant cell myeloma, heavy-chain myeloma, and light chain or Bence-Jones myeloma as well as leimyosarcoma.

In some embodiments, a cancer treated with a dosing regimen of method provided herein is a CD20+ cancer. In some embodiments, a cancer treated with a dosing regimen or method provided herein is a non-Hodgkin's lymphoma (NHL). In some embodiments, the cancer is diffuse large B cell lymphoma (DLBCL). In some embodiments, the cancer is a CD20+ DLBCL. In some embodiments, a cancer treated with a dosing regimen or method provided herein is relapsed and/or refractory (R/R). In some embodiments, the cancer is R/R to at least one prior therapy administered for the cancer. In some embodiments, the cancer is R/R DLBCL. In some embodiments, a subject treated with a dosing regimen or method provided herein has been previously treated with 1-3 lines of therapy for the cancer.

A CD47 blocking agent, anti-CD20/anti-CD3 bispecific antibody, and anti-CD20 antibody provided herein can be administered to the subject through any of the routes established for protein delivery, in particular, intravenous, intradermal and subcutaneous injection or infusion, or by oral or nasal administration. In some embodiments, the CD47 blocking agent, anti-CD20/anti-CD3 bispecific antibody, and anti-CD20 antibody are administered by intravenous administration.

Incorporated by reference herein for all purposes is the content of U.S. Provisional Patent Application No. 63/386,767, filed Dec. 9, 2022 and U.S. Provisional Patent Application No. 63/501,775, filed May 12, 2023.

The following examples of specific aspects for carrying out the present invention are offered for illustrative purposes only, and are not intended to limit the scope of the present invention in any way.

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### Sequences

Sequences provided herein are summarized in Table 1 below.

TABLE 1

| SEQ ID | | |
|---|---|---|
| NO: | Description | Sequence |
| 1 | SIRPa V2 | EELQVIQPDKSVSVAAGESAILHCTVTSLIPVGPIQWFRGAGP |
| | IgV domain | ARELIYNQKEGHFPRVTTVSESTKRENMDFSISISNITPADAG |
| | (residues | TYYCVKFRKGSPDTEFKSGA |
| | 32-137) | |
| 2 | SIRPa V2 | EEELQVIQPDKSVSVAAGESAILHCTVTSLIPVGPIQWFRGAG |
| | IgV domain + | PARELIYNQKEGHFPRVTTVSESTKRENMDFSISISNITPADA |
| | flanking | GTYYCVKFRKGSPDTEFKSGAGTELSVRAKPS |
| | residues | |
| | (residues | |
| | 31-148) | |
| 3 | Human IgG1 | DKTHTCPPCPAPELLGGPSVFLFPPKPKDTLMISRTPEVTCV |
| | Fc region, | VVDVSHEDPEVKFNWYVDGVEVHNAKTKPREEQYNSTYRV |
| | residues | VSVLTVLHQDWLNGKEYKCKVSNKALPAPIEKTISKAKGQPR |
| | 104-330 | EPQVYTLPPSRDELTKNQVSLTCLVKGFYPSDIAVEWESNG |
| | | QPENNYKTTPPVLDSDGSFFLYSKLTVDKSRWQQGNVFSC |
| | | SVMHEALHNHYTQKSLSLSPGK |
| 4 | Human IgG4 | ESKYGPPCPSCPAPEFLGGPSVFLFPPKPKDTLMISRTPEVT |
| | Fc region, | CVVVDVSQEDPEVQFNWYVDGVEVHNAKTKPREEQFNSTY |
| | residues 99- | RVVSVLTVLHQDWLNGKEYKCKVSNKGLPSSIEKTISKAKGQ |
| | 327 (P01861 | PREPQVYTLPPSQEEMTKNQVSLTCLVKGFYPSDIAVEWES |
| | UniProt) | NGQPENNYKTTPPVLDSDGSFFLYSRLTVDKSRWQEGNVF |
| | | SCSVMHEALHNHYTQKSLSLSLGK |
| 5 | Human IgG4 | ESKYGPPCPPCPAPEFLGGPSVFLFPPKPKDTLMISRTPEVT |
| | Fc region, | CVVVDVSQEDPEVQFNWYVDGVEVHNAKTKPREEQFNSTY |
| | residues 99- | RVVSVLTVLHQDWLNGKEYKCKVSNKGLPSSIEKTISKAKGQ |
| | 327, with the | PREPQVYTLPPSQEEMTKNQVSLTCLVKGFYPSDIAVEWES |
| | S228P | NGQPENNYKTTPPVLDSDGSFFLYSRLTVDKSRWQEGNVF |
| | mutation | SCSVMHEALHNHYTQKSLSLSLGK |
| 6 | Fusion of | EEELQVIQPDKSVSVAAGESAILHCTVTSLIPVGPIQWFRGAG |
| | SIRPa V2 | PARELIYNQKEGHFPRVTTVSESTKRENMDFSISISNITPADA |
| | IgV | GTYYCVKFRKGSPDTEFKSGAGTELSVRAKPSDKTHTCPPC |
| | extended | PAPELLGGPSVFLFPPKPKDTLMISRTPEVTCVVVDVSHEDP |
| | domain | EVKFNWYVDGVEVHNAKTKPREEQYNSTYRVVSVLTVLHQ |
| | (SEQ ID NO: | DWLNGKEYKCKVSNKALPAPIEKTISKAKGQPREPQVYTLPP |
| | 2) + IgG1 Fc | SRDELTKNQVSLTCLVKGFYPSDIAVEWESNGQPENNYKTT |
| | region (SEQ | PPVLDSDGSFFLYSKLTVDKSRWQQGNVFSCSVMHEALHN |
| | ID NO: | HYTQKSLSLSPGK |
| | 3)(TTI-621) | |
| 7 | Fusion of | EEELQVIQPDKSVSVAAGESAILHCTVTSLIPVGPIQWFRGAG |
| | SIRPa V2 | PARELIYNQKEGHFPRVTTVSESTKRENMDFSISISNITPADA |
| | IgV | GTYYCVKFRKGSPDTEFKSGAGTELSVRAKPSESKYGPPCP |
| | extended | PCPAPEFLGGPSVFLFPPKPKDTLMISRTPEVTCVVVDVSQE |
| | domain | DPEVQFNWYVDGVEVHNAKTKPREEQFNSTYRVVSVLTVL |
| | (SEQ ID NO: | HQDWLNGKEYKCKVSNKGLPSSIEKTISKAKGQPREPQVYT |
| | 2) + IgG4 Fc | LPPSQEEMTKNQVSLTCLVKGFYPSDIAVEWESNGQPENNY |
| | region with | KTTPPVLDSDGSFFLYSRLTVDKSRWQEGNVFSCSVMHEAL |
| | S228P | HNHYTQKSLSLSLGK |
| | mutation | |
| | (SEQ ID NO: | |
| | 5)(TTI-622) | |
| 8 | Linker | GGGGS |
| 9 | Linker | GTELSVRAKPS |
| 10 | Anti-CD20 | YSWIN |
| | HCDR1 | |
| 11 | Anti-CD20 | RIFPGDGDTDYNGKFKG |
| | HCDR2 | |
| 12 | Anti-CD20 | NVFDGYWLVY |
| | HCDR3 | |
| 13 | Anti-CD20 | RSSKSLLHSNGITYLY |
| | LCDR1 | |
| 14 | Anti-CD20 | QMSNLVS |
| | LCDR2 | |
| 15 | Anti-CD20 | AQNLELPYT |
| | LCDR3 | |
| 16 | Anti-CD20 | QVQLVQSGAEVKKPGSSVKVSCKASGYAFSYSWINWVRQA |
| | VH | PGQGLEWMGRIFPGDGDTDYNGKFKGRVTITADKSTSTAY |
| | | MELSSLRSEDTAVYYCARNVFDGYWLVYWGQGTLVTVSS |
| 17 | Anti-CD20 | DIVMTQTPLSLPVTPGEPASISCRSSKSLLHSNGITYLYWYLQ |
| | VL | KPGQSPQLLIYQMSNLVSGVPDRFSGSGSGTDFTLKISRVEA |
| | | EDVGVYYCAQNLELPYTFGGGTKVEIK |
| 18 | Anti-CD3 | TYAMN |
| | HCDR1 | |
| 19 | Anti-CD3 | RIRSKYNNYATYYADSVKG |
| | HCDR2 | |
| 20 | Anti-CD3 | HGNFGNSYVSWFAY |
| | HCDR3 | |
| 21 | Anti-CD3 | GSSTGAVTTSNYAN |
| | LCDR1 | |
| 22 | Anti-CD3 | GTNKRAP |
| | LCDR2 | |
| 23 | Anti-CD3 | ALWYSNLWV |
| | LCDR3 | |
| 24 | Anti-CD3 VH | EVQLLESGGGLVQPGGSLRLSCAASGFTFSTYAMNWVRQA |
| | | PGKGLEWVSRIRSKYNNYATYYADSVKGRFTISRDDSKNTL |
| | | YLQMNSLRAEDTAVYYCVRHGNFGNSYVSWFAYWGQGTL |
| | | VTVSS |
| 25 | Anti-CD3 VL | QAVVTQEPSLTVSPGGTVTLTCGSSTGAVTTSNYANWVQEK |
| | | PGQAFRGLIGGTNKRAPGTPARFSGSLLGGKAALTLSGAQP |
| | | EDEAEYYCALWYSNLWVFGGGTKLTVL |
| 26 | Anti-CD20 | DIVMTQTPLSLPVTPGEPASISCRSSKSLLHSNGITYLYWYLQ |
| | VL-CL (RK) | KPGQSPQLLIYQMSNLVSGVPDRFSGSGSGTDFTLKISRVEA |
| | (Polypeptide | EDVGVYYCAQNLELPYTFGGGTKVEIKRTVAAPSVFIFPPSD |
| | 1 of | RKLKSGTASVVCLLNNFYPREAKVQWKVDNALQSGNSQES |
| | glofitamab) | VTEQDSKDSTYSLSSTLTLSKADYEKHKVYACEVTHQGLSS |
| | | PVTKSFNRGEC |
| 27 | Anti-CD20 | QVQLVQSGAEVKKPGSSVKVSCKASGYAFSYSWINWVRQA |
| | VH- | PGQGLEWMGRIFPGDGDTDYNGKFKGRVTITADKSTSTAY |
| | CH1(EE)- | MELSSLRSEDTAVYYCARNVFDGYWLVYWGQGTLVTVSSA |
| | Fc(hole, | STKGPSVFPLAPSSKSTSGGTAALGCLVEDYFPEPVTVSWN |
| | PGLALA) | SGALTSGVHTFPAVLQSSGLYSLSSVVTVPSSSLGTQTYICN |
| | (Polypeptide | VNHKPSNTKVDEKVEPKSCDKTHTCPPCPAPEAAGGPSVFL |
| | 2 of | FPPKPKDTLMISRTPEVTCVVVDVSHEDPEVKFNWYVDGVE |
| | glofitamab) | VHNAKTKPREEQYNSTYRVVSVLTVLHQDWLNGKEYKCKV |
| | | SNKALGAPIEKTISKAKGQPREPQVCTLPPSRDELTKNQVSL |
| | | SCAVKGFYPSDIAVEWESNGQPENNYKTTPPVLDSDGSFFL |
| | | VSKLTVDKSRWQQGNVFSCSVMHEALHNHYTQKSLSLSP |
| 28 | Anti-CD3 | EVQLLESGGGLVQPGGSLRLSCAASGFTFSTYAMNWVRQA |
| | VH-CL | PGKGLEWVSRIRSKYNNYATYYADSVKGRFTISRDDSKNTL |
| | (Polypeptide | YLQMNSLRAEDTAVYYCVRHGNFGNSYVSWFAYWGQGTL |
| | 3 of | VTVSSASVAAPSVFIFPPSDEQLKSGTASVVCLLNNFYPREA |
| | glofitamab) | KVQWKVDNALQSGNSQESVTEQDSKDSTYSLSSTLTLSKAD |
| | | YEKHKVYACEVTHQGLSSPVTKSFNRGEC |
| 29 | Anti-CD20 | QVQLVQSGAEVKKPGSSVKVSCKASGYAFSYSWINWVRQA |
| | VH- | PGQGLEWMGRIFPGDGDTDYNGKFKGRVTITADKSTSTAY |
| | CH1(EE)- | MELSSLRSEDTAVYYCARNVFDGYWLVYWGQGTLVTVSSA |
| | Anti-CD3 | STKGPSVFPLAPSSKSTSGGTAALGCLVEDYFPEPVTVSWN |
| | VL-CH1- | SGALTSGVHTFPAVLQSSGLYSLSSVVTVPSSSLGTQTYICN |
| | Fc(knob, | VNHKPSNTKVDEKVEPKSCDGGGGSGGGGSQAVVTQEPSL |
| | PGLALA) | TVSPGGTVTLTCGSSTGAVTTSNYANWVQEKPGQAFRGLIG |
| | (Polypeptide | GTNKRAPGTPARFSGSLLGGKAALTLSGAQPEDEAEYYCAL |
| | 4 of | WYSNLWVFGGGTKLTVLSSASTKGPSVFPLAPSSKSTSGGT |
| | glofitamab) | AALGCLVKDYFPEPVTVSWNSGALTSGVHTFPAVLQSSGLY |
| | | SLSSVVTVPSSSLGTQTYICNVNHKPSNTKVDKKVEPKSCDK |
| | | THTCPPCPAPEAAGGPSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALGAPIEKTISKAKGQPREP |
| | | QVYTLPPCRDELTKNQVSLWCLVKGFYPSDIAVEWESNGQP |
| | | ENNYKTTPPVLDSDGSFFLYSKLTVDKSRWQQGNVFSCSV |
| | | MHEALHNHYTQKSLSLSP |

### EXAMPLES

In order that this invention may be better understood, the following examples are set forth. These examples are for purposes of illustration only and are not to be construed as limiting the scope of the invention in any manner.

### Example 1: A Phase 1b/2, Open-Label Study of PF-07901801 in Combination with Glofitamab after a Fixed, Single Dose of Obinutuzumab in Participants with Relapsed/Refractory Diffuse Large B Cell Lymphoma not Eligible for Stem Cell Transplantation

This is a multicenter, international, 2-component study (dose-escalation Phase 1b followed by a randomized Phase 2 part) of TTI-622 (maplirpacept) in combination with glofitamab after a single dose of obinutuzumab in participants with relapsed/refractory (R/R) diffuse large B cell lymphoma (DLBCL) who have completed at least 1 line of treatment (at least 1 containing an anti-CD20 therapy) and who are not candidates for high dose therapy/ASCT. In Phase 2, participants must have received 1 but not more than 2 prior lines of therapy.

Phase 1b will assess DLTs to select up to 2 doses of maplirpacept to be administered in combination with glofitamab in the randomized Phase 2 part of the study. The Phase 2 part is aimed to determine the recommended Phase 3 dose of maplirpacept to be administered in combination with glofitamab.

Approximately 70 participants will be enrolled in the study: approximately 20 participants in the Phase 1b part of the study and approximately 50 participants in Phase 2. Objectives of Phase 1b are to evaluate the safety and tolerability, preliminary anti-tumor activity, PK, PD of different doses of TTI-622 in combination with the glofitamab dose identified as the RP2D in the Phase 2 expansion of study NP30179 (NCT03075696) in participants with R/R DLBCL. Doses for the Phase 2 part of this study will also be selected. An escalation/de-escalation approach will be used in Phase 1b to identify the safe doses of TTI-622 when administered in combination with glofitamab. The dose levels to be explored are as follows:
Cohort 1: Each cycle (C) is 21 days. Prior to cycle 1, there is a lead-in phase. The lead-in phase is also referred to as cycle 0 (C0).
   C0: Day 1: Obinutuzumab (1000 mg); Day 8: glofitamab (2.5 mg); Day 15: glofitamab (10 mg)
   C1-C3: Day 1: glofitamab (30 mg) and TTI-622 (4 mg/kg or 300 mg); Day 8: TTI-622 (4 mg/kg or 300 mg); Day 15: TTI-622 (4 mg/kg or 300 mg).
   C4-C11: Day 1: glofitamab (30 mg) and TTI-622 (10 mg/kg or 600 mg)
   C12 and beyond: Day 1: TTI-622 (10 mg/kg or 600 mg).
Cohort 2: Each cycle (C) is 21 days. Prior to cycle 1, there is a lead-in phase. The lead-in phase is also referred to as cycle 0 (C0).
   C0: Day 1: Obinutuzumab (1000 mg); Day 8: glofitamab (2.5 mg); Day 15: glofitamab (10 mg)
   C1-C3: Day 1: glofitamab (30 mg) and TTI-622 (8 mg/kg or 600 mg); Day 8: TTI-622 (8 mg/kg or 600 mg); Day 15: TTI-622 (8 mg/kg or 600 mg).
   C4-C11: Day 1: glofitamab (30 mg) and TTI-622 (18 mg/kg or 1200 mg)
   C12 and beyond: Day 1: TTI-622 (18 mg/kg or 1200 mg).
Cohort 3: Each cycle (C) is 21 days. Prior to cycle 1, there is a lead-in phase. The lead-in phase is also referred to as cycle 0 (C0).
   C0: Day 1: Obinutuzumab (1000 mg); Day 8: glofitamab (2.5 mg); Day 15: glofitamab (10 mg)
   C1-C3: Day 1: glofitamab (30 mg) and TTI-622 (16 mg/kg or 1200 mg); Day 8: TTI-622 (16 mg/kg or 1200 mg); Day 15: TTI-622 (16 mg/kg or 1200 mg).
   C4-C11: Day 1: glofitamab (30 mg) and TTI-622 (28 mg/kg or 2400 mg)
   C12 and beyond: Day 1: TTI-622 (28 mg/kg or 2400 mg).

Optionally, in each of the above cohorts, TTI-622 will be administered in the fixed dose described above. Thus, for example, in Cohort 1, the dosing is as follows: C0: Day 1: Obinutuzumab (1000 mg); Day 8: glofitamab (2.5 mg); Day 15: glofitamab (10 mg); C1-C3: Day 1: glofitamab (30 mg) and TTI-622 (300 mg); Day 8: TTI-622 (300 mg); Day 15: TTI-622 (300 mg); C4-C11: Day 1: glofitamab (30 mg) and TTI-622 (600 mg) C12 and beyond: Day 1: TTI-622 (600 mg). In Cohort 2, the dosing is as follows: C0: Day 1: Obinutuzumab (1000 mg); Day 8: glofitamab (2.5 mg); Day 15: glofitamab (10 mg); C1-C3: Day 1: glofitamab (30 mg) and TTI-622 (600 mg); Day 8: TTI-622 (600 mg); Day 15: TTI-622 (600 mg); C4-C11: Day 1: glofitamab (30 mg) and TTI-622 (1200 mg); C12 and beyond: Day 1: TTI-622 (1200 mg). In Cohort 3, the dosing is as follows: C0: Day 1: Obinutuzumab (1000 mg); Day 8: glofitamab (2.5 mg); Day 15: glofitamab (10 mg); C1-C3: Day 1: glofitamab (30 mg) and TTI-622 (1200 mg); Day 8: TTI-622 (1200 mg); Day 15: TTI-622 (1200 mg); C4-C11: Day 1: glofitamab (30 mg) and TTI-622 (2400 mg); C12 and beyond: Day 1: TTI-622 (2400 mg).

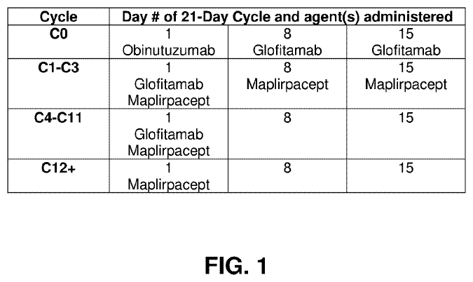

A schematic showing the outline of dosing for the different cycles (common to each of Cohorts 1-3) is shown in FIG. 1.

For each cohort, the glofitamab step-up dosing (2.5 mg/10 mg/30 mg) is intended to reduce the risk of cytokine-release syndrome (CRS). The sponsor may further escalate the TTI-622 dose or explore an intermediate dosing regimen based on emerging PK, PD, efficacy, and safety date.

To reduce the risk of glofitamab-induced cytokine release syndrome all study participants will be treated with a single dose of obinutuzumab 7 days prior to the first dose of glofitamab [or 21 days prior to cycle 1, day 1 (C1D1)]. Escalating doses of weekly glofitamab will be administered starting at 2.5 mg on day 8 and 10 mg at day 15 of C0. On C1D1 TTI-622 will be administered first; infusion with full dose of glofitamab (30 mg) will start at least 1 hour after the end of the TTI-622 infusion.

Optionally, during the lead-in phase (C0), obinutuzumab and glofitamab are administered but maplirpacept (TTI-622) is not administered. In other words, optionally, maplirpacept (TTI-622) is not administered during the lead-in phase.

In each cohort the selected dose and schedule of obinutuzumab and glofitamab are supported by safety data from Study NP30179 which investigated the step-up dosing regimen for glofitamab monotherapy. Pretreatment with a single dose of obinutuzumab has been shown to effectively deplete peripheral B-cells, reducing the risk of CRS that can result from rapid glofitamab mediated T-cell activation via interaction with B-cell targets in the circulation while glofitamab step-up dosing (2.5/10/30 mg) is an additional safety measure intended to reduce the risk of CRS, which frequency and severity is associated with higher initial glofitamab doses.

### Summary of Study Arms and Duration

Treatments will be administered in 21-day cycles. TTI-622 will be administered until disease progression or unacceptable toxicity while glofitamab will be administered up to and including cycle 11 or progressive disease or unacceptable toxicity whichever occurs first. TTI-622 infusion will precede glofitamab administration. Table 2 includes a summary of various aspects of the study.

TABLE 2

| | Study Intervention (21-day cycle) | | |
|---|---|---|---|
| Intervention Name | TTI-622 | Glofitamab | Obinutuzumab |
| Arm Name | Administered to | Administered to | Administered to |
| | all participants | all participants | all participants |
| Unit Dose | Cohort 1: 4 mg/kg or | All cohorts: | All cohorts: |
| Strength(s) | 300 mg QW C1-C3 | Step-up priming | 1000 mg minus 21 |
| | (Days 1, 8, and 15), | doses: | days from C1D1 |
| | 10 mg/kg or 600 mg | 2.5 mg minus 14 | (C0 day 1) |
| | Q3W C4 (Day 1) | days from C1D1 | |
| | and beyond | (C0 day 8) | |
| | Cohort 2: 8 mg/kg or | 10 mg minus 7 days | |
| | 600 mg QW C1-C3 | from C1D1 (C0 day | |
| | (Days 1, 8, and 15), | 15) | |
| | 18 mg/kg or 1200 mg | 30 mg Q3W C1-C11 | |
| | Q3W C4 (Day 1) | (Day 1) | |
| | and beyond | | |
| | Cohort 3: 16 mg/kg | | |
| | or 1200 mg QW C1- | | |
| | C3 (Days 1, 8, and | | |
| | 15), 28 mg/kg or | | |
| | 2400 mg Q3W C4 | | |
| | (Day 1) and beyond | | |
| Route of | IV infusion | IV infusion | IV infusion |
| Administration | | | |
| Duration of | Until disease | Up to 11 | Once |
| Administration | progression | cycles | |

### Study Population—Exemplary Inclusion Criteria

### Disease Characteristics:

Histologically confirmed and measurable relapsed and/or refractory, expected to express CD20, DLBCL not otherwise specified (NOS), primary mediastinal B-cell lymphoma (PMBCL) and transformed follicular lymphoma, T cell/histiocyte rich large B-cell lymphoma; Grade 3b follicular lymphoma; composite lymphoma with a DLBCL component with a subsequent DLBCL relapse.
Participants with evidence of histological transformation to DLBCL from an earlier diagnosis of low grade lymphoma (i.e. an indolent pathology such as follicular lymphoma) are eligible after relapse of at least one line of therapy for DLBCL
Measurable disease: at least 1 site of measurable PET-avid disease per Lugano 2014 classification (index lesion: >1.5 cm in the longest diameter for nodal, >1.0 cm in the longest diameter for extranodal disease).
Prior systemic treatment regimen: Must include an anti-CD20 containing regimen. Phase 1b: at least 1 prior line of systemic therapy. Phase 2: at least 1 but no more than 2 prior lines of systemic therapy. For both phases: induction, ASCT and consolidation will be considered one line of therapy; bridging therapy prior to CAR-T and CAR-T treatment is considered together as one line of therapy.

### Study Population—Exemplary Exclusion Criteria

### Prior/Concomitant Therapy:

Prior treatment with anti-CD47 and/or anti-SIRPa therapy and/or prior obinutuzumab or glofitamab containing regimen. Documented refractoriness to an Obinutuzumab monotherapy containing regimen.
Any prior systemic cancer treatment within 4 weeks before obinutuzumab infusion. Prior CAR-T within 90 days before obinutuzumab infusion, radiotherapy within 2 weeks or major surgery within 4 weeks prior to obinutuzumab infusion.

It is claimed:

1. A method of treating a cancer in a patient, the method comprising administering a combination therapy comprising a CD47 blocking agent and an anti-CD20/anti-CD3 bispecific antibody to the patient.

2. The method of claim 1, wherein the CD47 blocking agent comprises a CD47-binding form of human SIRPalpha (SIRPa).

3. The method of any one of claims 1-2, wherein the CD47 blocking agent comprises an Fc fusion protein comprising the IgV domain of human SIRPa variant 2 attached to an antibody Fc region (SIRPaFc fusion protein).

4. The method of claim 3, wherein the SIRPaFc fusion protein comprises the amino acid sequence of SEQ ID NO: 6 or SEQ ID NO: 7.

5. The method of any one of claims 1-4, wherein the anti-CD20/anti-CD3 bispecific antibody comprises a first antigen binding site that binds to CD3 and a second antigen binding site that binds to CD20, wherein the first antigen binding site comprises a VH and a VL, wherein the second antigen binding site comprises a VH and a VL, and wherein one or both of:
a) the first antigen binding site VH comprises the heavy chain CDR (HCDR) 1 of SEQ ID NO: 18, the HCDR2 of SEQ ID NO: 19, and the HCDR3 of SEQ ID NO: 20; and the first antigen binding site VL comprises the light chain CDR (LCDR) 1 of SEQ ID NO: 21, the LCDR2 of SEQ ID NO: 22, and the LCDR3 of SEQ ID NO: 23; and/or
b) the second antigen binding site VH comprises the heavy chain CDR (HCDR) 1 of SEQ ID NO: 10, the HCDR2 of SEQ ID NO: 11, and the HCDR3 of SEQ ID NO: 12; and the second antigen binding site VL comprises the light chain CDR (LCDR) 1 of SEQ ID NO: 13, the LCDR2 of SEQ ID NO: 14, and the LCDR3 of SEQ ID NO: 15.

6. The method of claim 5, wherein the first antigen binding site VH comprises a sequence that is at least about 95%, 96%, 97%, 98%, 99%, or 100% identical to the amino acid sequence of SEQ ID NO: 24, the first antigen binding site VL comprises a sequence that is at least about 95%, 96%, 97%, 98%, 99%, or 100% identical to the amino acid sequence of SEQ ID NO: 25, the second antigen binding site VH comprises a sequence that is at least about 95%, 96%, 97%, 98%, 99%, or 100% identical to the amino acid sequence of SEQ ID NO: 16, and/or the second antigen binding site VL comprises a sequence that is at least about 95%, 96%, 97%, 98%, 99%, or 100% identical to the amino acid sequence of SEQ ID NO: 17.

7. The method of any one of claims 1-6, wherein the anti-CD20/anti-CD3 bispecific antibody comprises a polypeptide comprising the sequence of SEQ ID NO: 26, a polypeptide comprising the sequence of SEQ ID NO: 27, a polypeptide comprising the sequence of SEQ ID NO: 28, and a polypeptide comprising the sequence of SEQ ID NO: 29.

8. The method of any one of claims 1-7, wherein the anti-CD20/anti-CD3 bispecific antibody is glofitamab.

9. The method of any one of claims 1-8, wherein the CD47 blocking agent and the anti-CD20/anti-CD3 bispecific antibody are administered to the patient for at least a first cycle, second cycle, third cycle, and fourth cycle, wherein each cycle is 21 days, wherein the CD47 blocking agent is administered on days 1, 8, and 15 of the first, second, and third cycle and on day 1 of the fourth cycle.

10. The method of any one of claims 1-9, wherein the CD47 blocking agent and the anti-CD20/anti-CD3 bispecific antibody are administered to the patient for at least a first cycle, second cycle, third cycle, and fourth cycle, wherein each cycle is 21 days, wherein the anti-CD20/anti-CD3 bispecific antibody is administered on day 1 of the first, second, third cycle and fourth cycle.

11. The method of any one of claims 1-10, wherein the CD47 blocking agent and the anti-CD20/anti-CD3 bispecific antibody are administered to the patient for at least a first cycle, second cycle, third cycle, and fourth cycle, wherein each cycle is 21 days, wherein the CD47 blocking agent is administered on days 1, 8, and 15 of the first cycle, second cycle, and third cycle and on day 1 of the fourth cycle, and wherein the anti-CD20/anti-CD3 bispecific antibody is administered on day 1 of the first, second, third, and fourth cycle.

12. The method of any one of claims 1-11, wherein the CD47 blocking agent and the anti-CD20/anti-CD3 bispecific antibody are administered to the patient for at least a lead-in cycle, a first cycle, second cycle, third cycle and fourth cycle, wherein the lead-in cycle precedes the first cycle, wherein each cycle is 21 days, wherein the anti-CD20/anti-CD3 bispecific antibody is administered on days 8 and 15 of the lead-in cycle, on day 1 of the first, second, third, and fourth cycle. Optionally, the CD47 blocking agent is not administered during the lead-in cycle.

13. The method of claim 12, further comprising administering an anti-CD20 monospecific antibody on day 1 of the lead-in cycle.

14. The method of any one of claims 1-13, wherein the CD47 blocking agent and the anti-CD20/anti-CD3 bispecific antibody are administered to the patient for at least a first cycle, second cycle, third cycle and fourth cycle, wherein each cycle is 21 days, wherein the CD47 blocking agent is administered at doses comprising 4 mg/kg, 8 mg/kg, 16 mg/kg, 300 mg, 600 mg, or 1200 mg on days 1, 8, and 15 of the first, second, and third cycle and at 10 mg/kg, 18 mg/kg, 28 mg/kg, 600 mg, 1200 mg, or 2400 mg on day 1 of the fourth cycle, and wherein the anti-CD20/anti-CD3 bispecific antibody is administered at doses comprising 30 mg on day 1 of the first, second, third, and fourth cycle, optionally wherein the CD47 blocking agent is administered at doses comprising 300 mg, 600 mg, or 1200 mg on days 1, 8, and 15 of the first, second, and third cycle and at 600 mg, 1200 mg, or 2400 mg on day 1 of the fourth cycle.

15. A method of treating a cancer in a patient, the method comprising administering a combination therapy comprising a CD47 blocking agent and an anti-CD20/anti-CD3 bispecific antibody to the patient, wherein the CD47 blocking agent is a SIRPaFc fusion protein comprising the amino acid sequence of SEQ ID NO: 7 (TTI-622), and wherein the anti-CD20/anti-CD3 bispecific antibody is glofitamab.

16. The method of claim 15, further comprising administering an anti-CD20 monospecific antibody to the patient.

17. A method of treating a cancer in a patient, the method comprising administering a combination therapy comprising a CD47 blocking agent and an anti-CD20/anti-CD3 bispecific antibody to the patient, wherein the CD47 blocking agent and the anti-CD20/anti-CD3 bispecific antibody are administered to the patient for at least a lead-in cycle, a first cycle, second cycle, third cycle and fourth cycle, wherein the lead-in cycle precedes the first cycle, wherein each cycle is 21 days, wherein the anti-CD20/anti-CD3 bispecific antibody is administered on days 8 and 15 of the lead-in cycle, and on day 1 of the first, second, third, and fourth cycle, wherein the method further comprises administering an anti-CD20 monospecific antibody on day 1 of the lead-in cycle, and wherein the CD47 blocking agent is administered on days 1, 8, and 15 of the first, second, and third cycle and on day 1 of the fourth cycle.

18. The method of any one of claims 1-17, wherein the cancer is a blood cancer or a solid tumor cancer.

19. The method of any one of claims 1-18, wherein the cancer is relapsed or refractory (R/R) diffuse large B cell lymphoma (DLBCL).

20. A CD47 blocking agent or anti-CD20/anti-CD3 bispecific antibody for use in the treatment of cancer according to the method of any one of claims 1-19.


SEQUENCE LISTING (29 sequences)

SEQ ID NO: 1 - protein, 106 aa, Homo sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 2 - protein, 118 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 3 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 345 aa, Synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 347 aa, Synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 5 aa, Synthetic construct
GGGGS

SEQ ID NO: 9 - protein, 11 aa, Synthetic construct
GTELSVRAKPS

SEQ ID NO: 10 - protein, 5 aa, Synthetic construct
YSWIN

SEQ ID NO: 11 - protein, 17 aa, Synthetic construct
RIFPGDGDTDYNGKFKG

SEQ ID NO: 12 - protein, 10 aa, Synthetic construct
NVFDGYWLVY

SEQ ID NO: 13 - protein, 16 aa, Synthetic construct
RSSKSLLHSNGITYLY

SEQ ID NO: 14 - protein, 7 aa, Synthetic construct
QMSNLVS

SEQ ID NO: 15 - protein, 9 aa, Synthetic construct
AQNLELPYT

SEQ ID NO: 16 - protein, 119 aa, Synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 17 - protein, 112 aa, Synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 18 - protein, 5 aa, Synthetic construct
TYAMN

SEQ ID NO: 19 - protein, 19 aa, Synthetic construct
RIRSKYNNYATYYADSVKG

SEQ ID NO: 20 - protein, 14 aa, Synthetic construct
HGNFGNSYVSWFAY

SEQ ID NO: 21 - protein, 14 aa, Synthetic construct
GSSTGAVTTSNYAN

SEQ ID NO: 22 - protein, 7 aa, Synthetic construct
GTNKRAP

SEQ ID NO: 23 - protein, 9 aa, Synthetic construct
ALWYSNLWV

SEQ ID NO: 24 - protein, 125 aa, Synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 25 - protein, 109 aa, Synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 26 - protein, 219 aa, Synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 447 aa, Synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 28 - protein, 232 aa, Synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 672 aa, Synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP